(19) United States
(12) Patent Application Publication
(10) Pub. No.: US 2025/0155433 A1
(43) Pub. Date: May 15, 2025

(54) FLUORESCENT SENSORS FOR DETECTION OF SIDEROPHORES AND OTHER MOLECULES PRODUCED BY BACTERIAL PATHOGENS

(71) Applicant: Kansas State University Research Foundation, Manhattan, KS (US)

(72) Inventors: Phillip E. KLEBBA, Manhattan, KS (US); Salete M. NEWTON, Manhattan, KS (US); Ashish KUMAR, Manhattan, KS (US); Taihao YANG, Manhattan, KS (US)

(21) Appl. No.: 18/722,025

(22) PCT Filed: Dec. 20, 2022
(86) PCT No.: PCT/US2022/082017

§ 371 Date: Jun. 20, 2024

Related U.S. Application Data
(60) Provisional application No. 63/291,735, filed on Dec. 20, 2021.

(51) Int. Cl. G01N 33/569; G01N 33/542; G01N 33/554

(52) U.S. Cl. CPC G01N 33/569; G01N 33/542; G01N 33/554; G01N 2333/195

(57) ABSTRACT

Methods, kits, and devices for detecting microbes in various samples. The methods, kits, and devices utilize a high affinity protein-based sensor, wherein the high affinity protein-based sensor comprises a high affinity binding protein and/or bacteria engineered with such a protein, with a detectable label that generates a detectable signal. The high affinity binding protein is specific for a microbe-associated compound secreted or produced by the target microbe When a biological sample containing or suspected of containing a microbe is contacted with the sensor, the presence of the microbe can be detected due to changes in the detectable signal in the assay over time, which correspond to interaction of the microbe-associated compound with the high affinity binding protein.

### CROSS-REFERENCE TO RELATED APPLICATIONS

The present application claims the priority benefit of U.S. Provisional Patent Application Ser. No. 63/291,735, filed Dec. 20, 2021, entitled FLUORESCENT SENSORS FOR DETECTION OF SIDEROPHORES AND OTHER MOLECULES PRODUCED BY BACTERIAL PATHOGENS, incorporated by reference in its entirety herein.

### BACKGROUND

### Field of the Invention

The present invention relates to high affinity protein-based assays for detecting and monitoring a variety of microbes by detecting molecules produced and secreted by the microbes.

### Description of Related Art

Siderophores are organic ligands that show specificity for iron. These small, high-affinity iron-chelating compounds are secreted by microorganisms, such as bacteria and fungi, and help the organism accumulate iron. Pathogenic bacteria secrete siderophores in the host environment, acquire their ferric complexes, and then utilize the metal for essential metabolic processes. They are also often directly correlated with virulence or pathogenicity of the organism. For example, prokaryotic siderophores facilitate colonization or pathogenesis of eukaryotes by liberating sequestered or stored host iron for bacterial utilization. Microbes produce and secrete a variety of other molecules, such as enzymes, toxins and small organic compounds, that help them interact with their environment. Methods and systems for quantitative and qualitative detection and monitoring of siderophores and other small molecule compounds secreted by microbes can provide an indirect way to detect and identify pathogenic agents in a variety of environments and types of samples.

### SUMMARY

Described herein are a variety of sensors for detecting siderophores, toxins, enzymes, and other small molecule compounds, particularly those secreted by microbes as a way to identify foreign pathogens in a sample and diagnose infection. By fluorescently labeling membrane receptors and binding proteins, we created sensors that detect, discriminate and quantify apo- and ferric siderophores and other molecules, including nutrients, metabolites, ligands, vitamins, enzymes and toxins that are associated with pathogens. The sensors can be designed with high specificity for specific target compounds which are associated with particular microbes, enabling identification of microbial families, genera, species, or strains and further enabling more targeted treatments. The sensors and assays described herein can be used in conjunction with the various sensors and assays described in U.S. Pat. No. 10,604,782, and U.S. Patent Publication No. US 2021/0263014, incorporated by reference herein, in their entireties, to further screen and identify suitable treatment compounds and conduct mechanistic studies of the detected microbes.

Thus, described herein are in vitro methods for detecting or characterizing a microbe in a biological sample. The methods generally comprise providing an assay solution comprising a high affinity protein-based sensor, wherein the high affinity protein-based sensor comprises a high affinity binding protein engineered with a detectable label that generates a detectable signal. The assay solution is exposed to an energy source to generate the initial detectable signal, and then the biological sample containing or suspected of containing the microbe is added to the assay solution, wherein the high affinity binding protein is specific for a microbe-associated compound secreted or produced by the microbe. The assay solution (exposed to the energy source) is observed and changes in the detectable signal in the assay solution over time are noted, wherein the changes correspond to interaction of the microbe-associated compound with the high affinity binding protein.

The present disclosure also contemplates kits for detecting a microbe in a biological sample. The kits generally comprise a vessel containing a high affinity protein-based sensor, wherein the high affinity protein-based sensor comprises a high affinity binding protein engineered with a detectable label that generates a detectable signal, wherein the high affinity binding protein is specific for a microbe-associated compound secreted or produced by the microbe. The kit also includes instructions for creating an assay solution with the high affinity protein-based sensor, exposing the assay solution to an energy source to generate the detectable signal; and instructions for adding a biological sample containing or suspected of containing the microbe to the assay solution and detecting changes in the detectable signal in the assay solution over time to detect the interaction of the microbe-associated compound with the high affinity binding protein (wherein the interaction indicates the presence of the microbe in the sample).

Also described herein are devices for detecting microbes in a biological sample. The devices generally comprise a multi-compartment microplate comprising a plurality of microwells, further comprising a high affinity protein-based sensor deposited in one or more of the microwells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic illustration of fluorescence of the detectable label in either engineered bacterial cells or engineered high affinity binding proteins and subsequent quenching in the presence of a target ligand.

FIG. 2 shows the binding results as detected by fluorescent intensity using engineered sensors for EcofepA, KpniroN, AbafepA, and PaefepA labeled with FM and then assayed for binding of different ferric catecholate siderophores The curves represent non-linear fits of the mean values to a single-site binding model using Grafit 6.0.12. The mean standard errors of the resulting K_{D} values from the fitted curves were: EcoFepA_A698C-FM, 17.3%; KpnIroNT210C-FM, 26.3%; AbaFepAS278C-FM, 21.1%; PaeFepA_S271C-FM, 22.4%.

FIG. 3 shows specific fluorescent binding results for FeAbn, FeYbt, FeAgn, vitamin B12, hemin, and FePvD by EcoIutA, KpnFyuA, AbaBauA, EcoBtuB, CcrHutA and PaeFpvA, respectively. The mean standard errors of the resulting K_{D} values from the fitted curves were: EcoIutA_T549C-FM, 13%; KpnFyuA_S631C-FM, 12%; AbaBauA_S385C-FM, 19%; EcoBtuB_S286C-FM, 16%; CcrHutA_A635C-FM, 18%; PaeFpvA_T660C-FM, 9.7%.

FIG. 4 shows the binding of ferric siderophores by (A) EcoFepB and (B) HsaSCN sensors for a spectrum of apo and ferric siderophores. The curves represent non-linear fits of the mean values to a single-site binding model using Grafit 6.0.12. The mean standard errors of the resulting K_{D} values from the fitted curves were: EcoFepB_S297C-FM, 23%; HsaScn_Q128C-FM, 21%.

FIG. 5 shows (A) the fluorescence intensity ratios (F/F₀) over time for four fluoresceinated sensor proteins (EcoFepA, KpnIroN, EcoIutA and KpFyuA) to assay for the presence of FeEnt, FeGEnt, FeAero and FeYbt, and the quenching pattern upon binding with the target compound′ (B) the fluorescence intensity ratios (F/F₀) over time for the KpnFyuA-FM sensor and quenching from sequential additions of spent ferrated media; and (C-F) graphs of the production of siderophores by E. coli and K. pneumoniae strains in iron-limited M9 and MOPS media.

FIG. 6 shows graphs of the fluorescence intensity ratios (F/F₀) over time of FeAbn, FePvd and B₁₂ uptake by CRE/ESKAPE pathogens.

FIG. 7 shows graphs verifying (A) the FeEnt and its degradation product (FeEnt*); and (B) the visible spectra of the purified iron complexes of FeEnt, FeEnt* and FeGEnt, each at 100 uM, differentiated them from one another.

FIG. 8 shows (A) a model of EcoIutA crystal structure generated by MODELLER of CHIMERA (UCSF), with the predicted locations of 6 site-directed Cys substitutions that we evaluated as sensors for FeAbn; (B) image of SDS-PAGE gel of cell envelope fractions from OKN359 derivatives; (C) fluorescence intensity ratios (F/F₀) over time for the sensors; (D) image from fluorescence scan of the same gel from (B); and (E) fluorescence intensity ratios (F/F₀) over time for EcoIutA_T549C.

FIG. 9 shows (A) a model of EcoBtuB crystal structure generated by MODELLER of CHIMERA (UCSF), with the predicted locations of 6 site-directed Cys substitutions that we evaluated as sensors for B12; (B) image of SDS-PAGE gel of cell envelope fractions from OKN359 derivatives; (C) fluorescence intensity ratios (F/F₀) over time for the sensor; (D) image from fluorescence scan of the same gel from (B); and (E) fluorescence intensity ratios (F/F₀) over time for EcoBtuB_S286C-FM.

FIG. 10 shows (A) a model of KpnFyuA crystal structure generated by MODELLER of CHIMERA (UCSF), with the predicted locations of 5 site-directed Cys substitutions that we evaluated as sensors for FeYbt; (B) image of SDS-PAGE gel of cell envelope fractions from OKN359 derivatives; (C) fluorescence intensity ratios (F/F₀) over time for the sensor; (D) concentration dependence of the extent of quenching to a single site binding model; and (E) image from fluorescence scan of the same gel from (B).

FIG. 11 shows (A) a model of AbaBauA crystal structure generated by MODELLER of CHIMERA (UCSF), with the predicted locations of 4 site-directed Cys substitutions that we evaluated as sensors for FeAgn; (B) image of SDS-PAGE gel of cell envelope fractions from OKN359 derivatives; (C) fluorescence intensity ratios (F/F₀) over time for the sensor; (D) image from fluorescence scan of the same gel from (B).

FIG. 12 shows crystal structures and Cys substitutions for EcoFepB (panel A) and HsaSCN (panel D); images of SDS-PAGE gels, stained with Coomassie blue for pEcoFepB T297C-6H (panel B) or pHsaSCNQ128C-6H (panel E); and fluorescence scans via UV illumination of the same gels (panel C and panel F, respectively).

FIG. 13 shows the results from Example 2, including (A) images of fluorescent scans of SDS-PAGE gels confirming fluorescent labeling of Cys substitutions in E. coli FepA; and (B) analysis of fluorescence quenching during FeEnt binding to EcoFepA-A698C-FM over time.

FIG. 14 shows the results from Example 3, including (A) the crystal structure of EcoPbp2 and Cys mutations; (B) SDS-PAGE gel image to confirm FM labeling; and (C) graph of fluorescence intensity change (1-F/F₀) at different concentrations of ampicillin.

### DETAILED DESCRIPTION

This invention represents a new concept in methods to recognize, discriminate and quantify microbes and/or foreign pathogens, particularly bacteria or fungi, in various environments, including research samples, clinical samples, or food samples, based upon detecting siderophores and other small molecule compounds that are produced and/or secreted by the bacteria or fungi, and typically can even be correlated with proliferation and/or pathogenicity of the organism. For example, siderophores solubilize iron for utilization by microorganisms, and facilitate colonization or infection of eukaryotes by liberating host iron for bacterial uptake. Accordingly, their presence and quantity in a sample indicates the presence (and potential pathogenicity) of the corresponding microbe. These compounds are referred to herein as “microbe-associated compounds.” The invention leverages engineered high affinity protein-based sensors and associated assays for detecting these microbes and foreign pathogens in a variety of samples in both qualitative and quantitative ways. These sensors can be used to detect microbes in a variety of biological samples, including bodily fluids (e.g., blood, plasma, serum, urine, exhaled breath condensate, sputum, bronchoalveolar lavage fluid, sweat, saliva, cervicovaginal fluid, rectal secretion, and tears), “food and drink samples” which is used herein to refer to food and drink products, ingredients, and pre-mixes per se, as well as handling and/or processing equipment therefor (e.g., milk and dairy products, fresh and processed meats, food ingredients and/or pre-mixes, bins, mixers blenders, grinders, choppers, cutters, storage tanks, mixing tanks, pipes, etc.), environmental samples (e.g., air, soil and water samples, plant materials and tissues, bioremediation sites), as well as research materials (e.g., water, cell culture media, pharmaceutical products). The samples can be the material itself, typically suspended in a buffer, or may be a sample taken, e.g., via swab, of a surface or material (e.g., equipment surface, oral mucosa swab, etc.) to be tested for presence of a microbe (which swab is then contacted with a buffer to prepare a test solution). It will be appreciated that although the sensors are exemplified herein for detecting unwanted pathogens, they are also useful for general surveying of potential microbes present in a sample (including positive ones), such as in taking a census of gut or oral health (e.g., to survey the gut or oral microbiome), assessing a microbiome panel for a person, animal, place, or thing, and provide general information regarding various microbes that may be present in a sample.

Examples of microbe-associated siderophores that can be detected include ferric catecholates (e.g., enterobactin, degraded enterobactin, glucosylated enterobactin, dihydroxybenzoate, dihydroxybenzoyl serine, cefidericol, MB-1), ferric hydroxamates (e.g., ferrichromes, aerobactin), mixed iron complexes (e.g., yersiniabactin, acinetobactin, pyoverdine), and porphyrins (e.g., hemin, vitamin B12).

Different microbes produce different siderophores, and the exquisite specificity of the sensors, combined with their potent sensitivity, allows the detection, discrimination and quantification of these microbe-associated compounds, which in turn provides information about the microbes in the sample.

Examples of other microbe-associated molecules that can be detected, include cell envelope or secreted proteins that confer antibiotic resistance, like penicillinases, carbapenemases, and other enzymes that degrade antibiotics. Examples of microbe-associated toxins that can be detected include endotoxins like lipopolysaccharides, and exotoxins like pertussis, shiga, botulinum and anthrax toxins.

Advantageously, the methods allow the quantification of microbe-associated compounds, that strongly correlate with the pathogenicity of those organisms. They are of potential value in a variety of situations and venues, including clinical medicine, agricultural and food applications, and laboratory research. No comparable technology exists with similar sensitivity, accuracy, and ease of use.

Thus, described herein are fluorescence assays for detection of specific microbial organisms in various biological samples through detection of specific microbe-associated compounds. The platform involves labeling receptor proteins with a fluorescent moiety or detectable label, and expressing the mutant proteins in a host organism (e.g., E. coli) to create the sensor platform which can then permit monitoring of recognition/binding by the microbe-associated compounds as well as transport of the microbe-associated compounds.

In general, the sensors comprise a high affinity binding protein engineered with a detectable label (i.e., molecular reporter moiety). Exemplary detectable labels include fluorescent dyes or fluorophores. Once labeled, these sites become reporter groups (i.e., fluorescent probes) that convey information about the presence or absence of microbe-associated compounds in a sample. Hence the experimental system spectroscopically reports on when a given microbe-associated compound (ligand) binds to its sensor (binding protein), thus quenching the detectable label, the extent of which can be spectroscopically measured and associated with a corresponding degree of microbial activity in the sample.

As used herein, references to “high affinity” binding protein means a protein with a strong affinity for its target ligand (which in this case is a microbe-associated compound), characterized by a binding reaction with an equilibrium dissociation constant (K_{D}) value in the nanomolar range (<1000 nM) or below, preferably 300 nM or less, preferably 100 nM or less, more preferably 50 nM or less, and even more preferably 15 nM or less (0.1-15 nM), and even more preferably 10 nM or less (0.1-10 nM). The dissociation constant corresponds to the ligand molar concentration at which half of the proteins are occupied at equilibrium, that is, the molar concentration of ligand at which the molar concentration of protein with bound ligands equals the molar concentration of protein with no ligand bound. The smaller the dissociation constant, the more tightly bound the ligand is, and the higher the affinity between ligand and protein. For example, a ligand with a nanomolar (nM) dissociation constant binds more tightly to a particular protein than a ligand with a micromolar (μM) dissociation constant. In general, such proteins are typically part of bacterial transport systems for uptake of various critical elements, nutrients, etc. from medium into the cell. However, it should be noted that the same technology efficiently functions, albeit with different advantages and disadvantages, and therefore, applications, when utilizing lower affinity, binding proteins with micromolar or millimolar K_{D} values.

In one or more embodiments, the engineered high affinity binding protein is a bacterial membrane protein (e.g., Gram (−) outer membrane transporter or Gram (+) lipoprotein anchored on the membrane), which are also sometimes referred to a ligand-gated porins (LGP), with high affinity for binding particular ligands (microbe-associated compounds) in solution. In one or more embodiments, the sensor is provided in the form of the engineered high affinity binding protein itself. Any suitable binding protein can be used, with exemplary high affinity binding proteins being derived from TonB-dependent ligand-gated porins (LGPs) of Escherichia coli (Fiu, FepA, Cir, FhuA, IutA, BtuB), Klebsiella pneumoniae (IroN, FepA, FyuA), Acinetobacter baumannii (PiuA, FepA, PirA, BauA), Pseudomonas aeruginosa (FepA, FpvA), and Caulobacter crescentus (HutA), as well as from a periplasmic E. coli binding protein (FepB), and from a human serum binding protein (siderocalin), a component of the innate immune system that recognizes many siderophores. As exemplified in the working examples, PCR or other suitable techniques can be used to clone a binding protein of interest from the chromosomes of a particular bacterial species.

In one or more embodiments, the sensor is provided in the form of a transport-deficient bacterial cell comprising the engineered high affinity binding protein expressed on its outer membrane surface. Exemplary bacteria for use to develop sensors include Escherichia coli, Acinetobacter baumannii, Klebsiella pneumoniae, Pseudomonas aeruginosa and other microorganisms with high affinity for particular ligands. The bacteria can be engineered to express the particular high affinity binding protein on its outer membrane surface for detection. In general, this is accomplished using standard plasmid transfer systems in which the desired sequence encoding for the desired (possibly foreign) high affinity bind protein is inserted under the control of appropriate regulatory sequences (e.g., signal and promoters) to direct secretion and assembly of the high affinity protein in the outer membrane of the sensor bacteria.

In general, when intact bacteria are used, they are transport-deficient bacteria, particularly for the ligand of interest. The sensor bacterium may have a native deficiency in its ability to transport the particular ligand of interest, or may be engineered to have a defective transport mechanism for the target. In other words, in the context of the current disclosure, the cell-based assays utilize sensor bacteria capable of binding the target ligand with specificity, but incapable of transport and uptake of the analyte. As such, the bacteria may be considered physiologically “inert,” as lacking active transport functions at least for the target analyte. Examples include TonB-deficient bacteria lacking active iron transport capabilities in iron assays.

Regardless of the embodiments, the high affinity binding protein is genetically-engineered or mutated, wherein the mutation comprises or consists of at least one amino acid residue of the native (chromosomal) protein substituted with a cysteine residue to generate the mutated protein. In one or more embodiments, the cysteine substitution results in a “single accessible cysteine” residue on the folded protein, which means that the location of the inserted cysteine residue is presented outwardly from the surface of the bacterial membrane or folded protein, such that it is “accessible” to react for labeling.

Accordingly, exemplary amino acid residues to be targeted for substitution should meet at both of the following criteria: (1) the targeted amino acid forms part of an outer loop of the native protein (so that the mutated cysteine residue will be outwardly presented); and (2) mutation of the targeted amino acid does not impair the ligand binding capabilities of the protein. In general, the targeted amino acid residues for mutation (and subsequent labeling) are those in the native protein with small side chains that are localized on the cell surface or outwardly presented on the folded protein. It will be appreciated that a protein native to the engineered bacteria may be altered in this manner (homologous protein modification). Alternatively, the engineered bacteria may be modified to express a non-native protein (e.g., a heterologous binding protein from a different bacterial species) that has been modified to include the cysteine substitution for labeling (again provided that the binding capabilities are not impaired) (plasmid-mediated modifications).

The specific mutated residue and target residue position will depend on the bacteria or protein used for the sensors. For FepA, exemplary native amino acids for targeted substitution include serine, glutamic acid, aspartic acid, alanine, and threonine, although other amino acids may also be suitable for Cys substitution. For example, when E. coli is the sensor bacteria, suitable native FepA residues targeted for cysteine substitution (and subsequent labeling) include residues 216, 271, or 698. Likewise, exemplary residues for substitution in A. baumannii FepA sensors include residues 278, 561, or 664. Exemplary residues for substitution in K. pneumoniae FepA sensors include 210. Exemplary residue substitutions in E. coli for FhuA sensors include residues 396. Exemplary residue substitutions in Hpb2 protein-based sensors include 154.

It will be appreciated that the techniques illustrated in the working examples can be used to identify additional residues for substitution (and subsequent labeling) in other proteins and bacteria. Various known approaches can be used to engineer the proteins and bacteria to obtain the cysteine substitution. Further, it will be appreciated that wild type proteins and bacteria can be used to develop the sensors, the proteins and bacteria sensors may include other modifications in addition to the cysteine substitution described here, with the proviso that the native target ligand binding capabilities of the bacteria or protein are not impaired.

As noted, the high affinity binding protein is engineered for labeling with a suitable reporter moiety. In general, this involves incubating the engineered protein (or bacteria), having the available engineered cysteine residue, with the selected fluorophore for a sufficient period of time, and under sufficient conditions to react with the free cysteine residue on the engineered protein. Preferably, the fluorophore is covalently attached to the engineered cysteine residue. In one or more embodiments, the fluorescent dye is a chemically-reactive derivative of fluorescein. Exemplary fluorophores include maleimide fluorophores, such as fluorescein maleimide, CPM (7-Diethylamino-3-(4′-Maleimidylphenyl)-4-Methylcoumarin), Alexa Fluors 488, 546, 555, 647, 680, and 720 and the like. In one or more embodiments, the fluorophore is incubated with the engineered protein (or bacteria) in phosphate buffer for at least about 5 min., and preferably from about 5 min. to about 15 min., at a temperature of from about 0° C. to about 37° C., and a pH ranging from about 6.5 to about 6.8. In this manner, site-directed Cys substitutions in receptor proteins allow their selective alkylation by maleimide fluorophores. The labeling reaction is then quenched, and the labeled proteins or cells are washed. Additional approaches to achieve fluorescent labeling are discussed in the working examples.

The labeled cells or proteins are then suspended in an aqueous solution. Exemplary aqueous solutions include solutions that are non-toxic to cells, and help maintain a neutral pH of the reaction solution in order not to destroy the sensor protein or cell and maintain the osmolarity of the cells. For example, buffer solutions including buffered saline solutions, such as phosphate buffered saline (PBS), are preferred aqueous solutions for used in the invention.

In one or more embodiments, the engineered high affinity protein-based sensors are then used directly in the assay or preserved for later use. In one or more embodiments, the engineered high affinity protein-based sensors are suspended in a buffer solution along with a cryoprotectant, such as DMSO and/or glycerol, followed by cryopreservation of the labeled cells for future use. In one or more embodiments, cell-based sensors can be cryopreserved for at least about 2 weeks and conceivably indefinitely before use in the assay. Likewise, because the sensor proteins can be expressed from plasmids, engineered sensor strains that host them may be centrally stored and distributed to any interested researcher.

These specific fluorescent sensors can detect, discriminate, and quantify the presence of numerous different microbe-associated compounds in a sample. Simple tests with these sensors may diagnose the presence of particular bacterial pathogens in a variety of experimental, clinical or food and drink samples, or predict bacterial tissue tropism during infections.

In one or more embodiments, the assay comprises preparing a reaction solution comprising a plurality of engineered sensor proteins or sensor bacteria having affinity for a particular microbe-associated compound. The reaction solution generally further comprises at least one nutrient (i.e., carbon source) dispersed in the aqueous solution to support bacterial cell functioning in the reaction solution. Exemplary nutrients will depend upon the particular sensor bacteria used, and include glucose, sodium acetate, succinate, and the like.

The reaction solution is formed in a reaction vessel. In one or more embodiments, the reaction vessel is cuvette or a microwell. In one or more embodiments, the reaction vessel has a working volume of up to about 3 mL, and in some embodiments less than about 3 mL, less than about 2 mL, less than about 1 mL, and even less than about 0.5 mL. It will be appreciated that the “working volume” of the reaction vessel is less than its total volume, and refers to the recommended volume to be utilized in individual vessels for the reaction solution, to avoid overflowing the vessel's total capacity. Thus, the reaction solution volume in the inventive assay will comprise less than about 3 mL total solution, less than about 2 mL total solution, less than about 1 mL total solution, or even less than bout 0.5 mL total solution. Advantageously, the assays can be conducted in even smaller volumes, usually from about 25 μL to 300 μL, such as in a microplate or microfluidics vessel.

In one or more embodiments, assays can be run from initial mixtures of 0.5 mL to 1 mL vials with testing of 200 μL aliquots. In one or more embodiments, the assay can be run in cuvettes or microwells and read with a spectrometer or fluorometer. In one or more embodiments, about 1 mL to 2 mL of non-interfering buffer or solvent can be added to a test container (e.g., well, cuvette, etc.), followed by about 50 μL of sensor, and about 50 μL to about 100 μL of sample (suspected of containing microbes). The reaction mixture can be allowed to react for about 15 min or less and then results can be read with a fluorometer (where the fluorescence quenching is observable).

It will be appreciated that an advantage of the inventive assays is miniaturization of the biochemical system to function in small volumes (≤300 μL), which allows the use of multi-compartment microtiter plates (e.g., 96- or 384-well plates). In one or more embodiments, the microplate reaction vessel has a working volume of 300 μL or less. In one or more embodiments, the reaction vessel has a working volume of 75 μL or less.

In one or more embodiments, the testing is carried out in a microwell that is part of a multi-well array in a microplate (aka microtiter plate). The plates may include 96, 384, or 1536, etc., wells disposed across the surface of the plate substrate, and generally arranged in a rectangular matrix. In general, the diameter, depth and spacing of the microwells in the plate can vary.

This adaptation of the methodology makes it compatible with high-throughput screening of samples for foreign microbes. In one or more embodiments, pre-loaded microtiter plates could be provided where the wells are pre-loaded with sensors for designated microbes. A user simply needs to add the amount of sample to each pre-loaded well and then read the results. The wells can be pre-loaded with sensors in different combinations, e.g., different sensors in each well, multiple rows of sensors, with each row having a different plurality of sensors (one type per well). The sensors can be used to detect various molecules made by or secreted from different target pathogens, such as the siderophores exemplified herein, as well as enzymes, and the like.

The total sensor protein or cell density in the reaction solution in each reaction vessel may vary. In general, low turbidity (dilute) reaction solutions are desired. In other words, the total sensor protein or cell density is preferably adjusted such that an adequate quantity of sensor proteins or cells are present in each reaction vessel to generate a detectable quenching reaction; however, an excess concentration of sensor proteins or cells in the reaction solution is counterproductive due to physically blocking of the detectable signal from the reaction vessel. In one or more embodiments, total cell density in the reaction solution ranges from about 5×10⁶ to about 5×10⁷ cells/ml, preferably from about 5×10⁶ to about 3×10⁷ cells/ml, and more preferably from about 5×10⁶ to about 2.5×10⁷ cells/ml. In one or more embodiments, the total cell density is about 2.5×10⁷ cells/ml or less. Optical density of the reaction solution can also be used to adjust the reaction solution cell concentration. Advantageously, the inventive methods can be used with cryopreserved sensor cells. It will be appreciated that in the case of cryopreserved cells, the cells will be thawed and subjected to appropriate post-thawing protocols before use in the assay (e.g., centrifugation and washing to remove cryoprotectant).

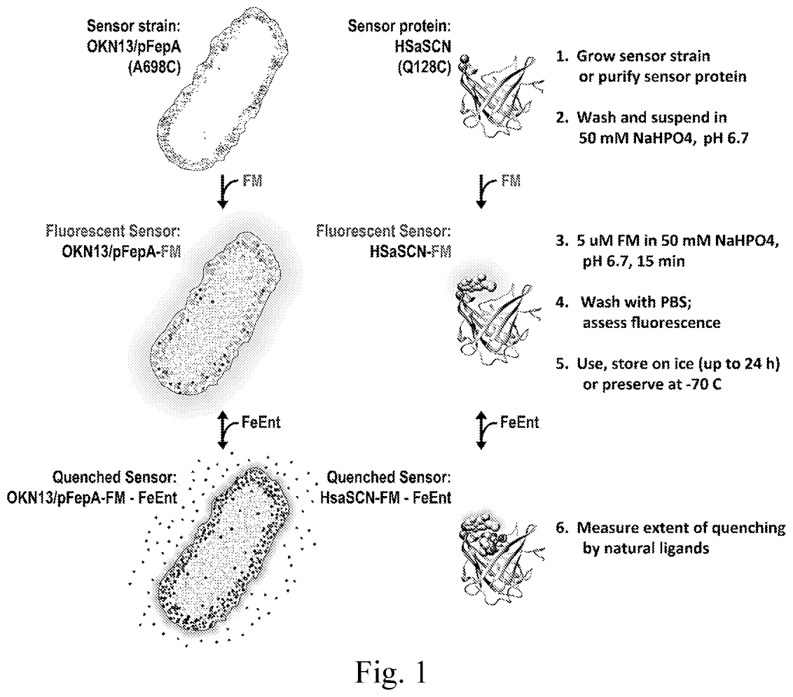

Regardless of the embodiment, once the sample containing or suspected of containing the microbes of interest is added, the assay is then monitored over time. More specifically, the detectable label on the engineered high affinity protein generates a signal that can be detected spectroscopically, and which changes over time depending upon the interaction (or lack thereof) of the ligand and the sensor protein or bacteria. In general, the assay is observed or the plate is read before and after the addition of the test sample to the reaction solution. In particular, in the case of fluorescent labels, the assay solution is excited at the appropriate wavelength(s) to generate an observable fluorescence from the sensors in the solution, as illustrated in FIG. 1. After the sample is added to the assay solution, it is then observed for potential fluorescence quenching that occurs upon binding of the microbe-associated compounds (if preset in the sample) to the sensor. This mechanism is also illustrated in FIG. 1. Using various approaches for calibrating the sensors and data analyses, the fluorescence quenching data can indicate both the presence and quantity of the microbe-associated compounds, if present in the sample. Notably, sensors that possess high affinity for a single ligand can unambiguously detect, identify, and quantify that compound in even complex samples.

For example, a first fluorescence reading is taken of the reaction solution before addition of the sample. A second fluorescence reading is then taken of the reaction solution over time after addition of the sample to monitor the extent of quenching. Various instruments are available for detecting the fluorescence signal, including commercially-available fluorometers.

The assays can be optimized to reduce interference with impurities in the reaction solution, including unintentional or incidental amounts of metal complexes and the like introduced during the process or produced by the sensor cells themselves. For example, the cells can be washed before analysis to remove any siderophores that may have been produced by the cells in response to iron-deficient conditions. Moreover, it is desirable to use buffer systems that are substantially free of adventitious metal complexes and/or metabolites to constrain biosynthesis by microbes that may be present in the samples.

Using the decoy sensors we defined the specificities, binding affinities and uptake rates of the transporters, sensitively detected the bacterial products in a different types of samples, and monitored ferric siderophore acquisition by microbial pathogens, all without the need to genetically engineer the infectious organisms under investigation. These data may be diagnostic for the presence of particular bacterial pathogens in experimental samples.

The platform can be used as direct sensors of the microbe-associated compounds, or can be mixed in the assay with other known organisms or compounds to provide indirect information about the activity of the other organism in the sample. These engineered sensor organisms can be stored frozen/cryopreserved, and then for use they can be thawed, mixed with a sample potentially containing the target, and the changes in the detectable moiety can be observed.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

### Introduction

Eukaryotic hosts and prokaryotic pathogens compete for iron, which influences the outcome of bacterial infections. In aqueous solutions, iron oxidizes and polymerizes as ferric oxyhydroxide (Fe(OH)ₙ), the low solubility of which (K_{SP}=2.79×10⁻³⁹) antagonizes its biological utilization. But, microbial siderophores chelate Fe³⁺ with such high affinity (Kₐ=10²⁰-10⁵⁰) that they release it from precipitates as hexacoordinate complexes that are available for assimilation. Conversely, humans and animals produce serum transferrin (TF), lactoferrin (LF) and cellular ferritin (FTN), that bind, sequester or store iron. The vertebrate innate immune system also actively inhibits siderophore-mediated iron acquisition by producing siderocalin (SCN; also called NGAL or lipocalin 2). Nevertheless, aposiderophores can capture Fe³⁺ from the binding proteins, and Gram (−) bacteria internalize ferric siderophores, heme, and other porphyrins through TonB-dependent outer membrane (OM) receptor proteins. Consequently, siderophore biosynthesis and TonB-dependent iron uptake promote colonization of the animal gut. Prokaryotic iron acquisition pathways are also a potential vulnerability, in that their disruption inhibits bacterial growth, reduces virulence and may eliminate pathogenesis.

In Gram (−) cells Fe³⁺ acquisition begins when cell surface receptors recognize and bind iron complexes. We designate these outer membrane (OM) proteins as ligand-gated porins (LGP) because they contain a 22-stranded β-barrel that places them in the porin superfamily, and because ligand binding triggers the TonB-dependent opening of their normally closed channels. Nevertheless, LGP are distinct from diffusive porins: they do not contain open transmembrane pores like those of OmpF or LamB. The C-terminal channels of LGP are occupied by an N-terminal, ˜150-residue globular domain, that regulates ligand uptake by interactions with the energy-dependent inner membrane (IM) protein TonB. All LGP-mediated Fe³⁺ transport requires the actions of TonB, that anchors in the IM, but spans the periplasm to associate with proteins in the OM.

Acquisition of certain ferric siderophores may promote pathogenesis and the invasion of specific host tissues. For example, the production of yersiniabactin (Ybt) by Yersinia pestis correlates with the development of bubonic or pneumonic plague. A variety of Gram (−) bacteria produce aerobactin (Abn) and utilize ferric aerobactin (FeAbn), that enhances their virulence, invasiveness and tissue tropism. Similarly, elaboration of the catecholate enterobactin (Ent) and uptake of ferric enterobactin (FeEnt) facilitates bacterial colonization of eukaryotic hosts. Glycosylation of Ent (GEnt) by the IroA system of K. pneumoniae and other pathogens, reduces its neutralization by SCN, enhancing bacterial infection of specific tissues (for example, by K. pneumoniae). These relationships between specific siderophores and infectious diseases, as well as the patterns of siderophore expression and transport during colonization of host tissues, underscore the potential value of biochemical tools that may characterize or explicate such phenomena. Sensors of apo- and ferric siderophores, reported herein, report kinetic and thermodynamic data on prokaryotic iron acquisition systems, and may provide diagnostic information about the nature and virulence of bacterial infections. LGP discriminate among >500 iron complexes, so their fluorescent derivatives may sensitively inform about which siderophores are present in the microenvironment, or what their concentrations are in clinical samples, or how their utilization relates to pathogenicity. Toward those ends, the production of a fluorescent LGP (FLGP) in a transport-deficient, AtonB host creates a “decoy” sensor cell, that detects and quantifies ligands in experimental suspensions or solutions. We used this strategy to produce 18 FLGP sensors, and 2 fluorescent binding protein (FBP) sensors, from siderocalin and EcoFepB. Collectively, they detected siderophore biosynthesis, measured siderophore concentrations and monitored ferric siderophore uptake by cells of interest.

### Results and Supplemental Information

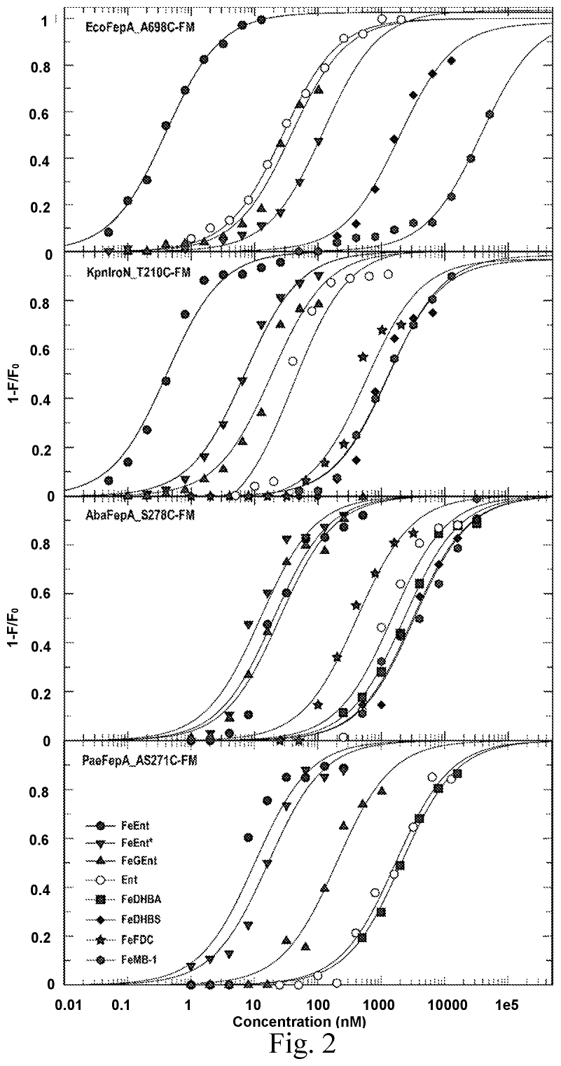

The results are shown in the Figures and discussed in more detail below. FIG. 2 shows broad recognition of ferric catecholates by FepA orthologs. EcofepA, KpniroN, AbafepA and PaefepA were cloned in pITS23, mutagenized to introduce single Cys residues, labeled with FM and then assayed for binding of different ferric catecholate siderophores (see also Table 1). The comparisons revealed a hierarchy of affinities for mono- and tricatecholate iron complexes that was different among EcoFepA and its homologs in the 3 ESKAPE bacteria, that roughly mimicked their extents of sequence identity. For each data point we made 3 determinations of quenching in response to the addition of different concentrations of an iron complex. The curves represent non-linear fits of the mean values to a single-site binding model using Grafit 6.0.12. The mean standard errors of the resulting K_{D} values from the fitted curves were: EcoFepA_A698C-FM, 17.3%; KpnIroNT210C-FM, 26.3%; AbaFepAS278C-FM, 21.1%; PaeFepA_S271C-FM, 22.4%.

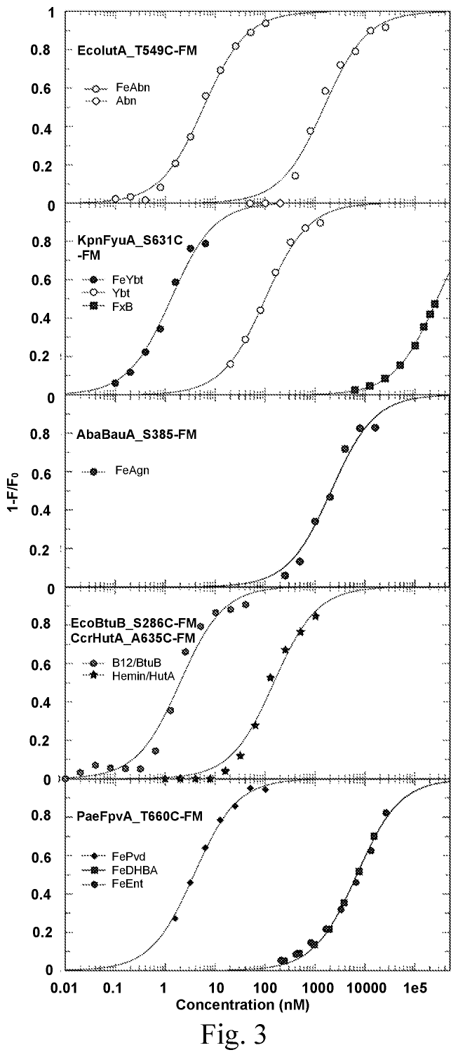

FIG. 3 shows specific recognition of FeAbn, FeYbt, FeAgn, B₁₂, hemin, and FePvD by EcoIutA, KpnFyuA, AbaBauA, EcoBtuB, CcrHutA and PaeFpvA, respectively. Structural genes of each of the LGP were cloned in pITS23, mutagenized to introduce single Cys residues, labeled with FM and then assayed for binding of different hydroxamate and mixed-chelation siderophores (see also Table 1). For each data point we made 3 determinations of quenching in response to the addition of different concentrations of an iron complex. The curves represent non-linear fits of the mean values to a single-site binding model using Grafit 6.0.12. The mean standard errors of the resulting K_{D} values from the fitted curves were: EcoIutA_T549C-FM, 13%; KpnFyuA_S631C-FM, 12%; AbaBauA_S385C-FM, 19%; EcoBtuB_S286C-FM, 16%; CcrHutA_A635C-FM, 18%; PaeFpvA_T660C-FM, 9.7%. The experiment revealed nearly exclusive binding of all the metal complexes by their respective LGP receptor proteins. KpnFyuA also weakly bound FxB, and PaeFpvA weakly bound FeEnt and FeDHB.

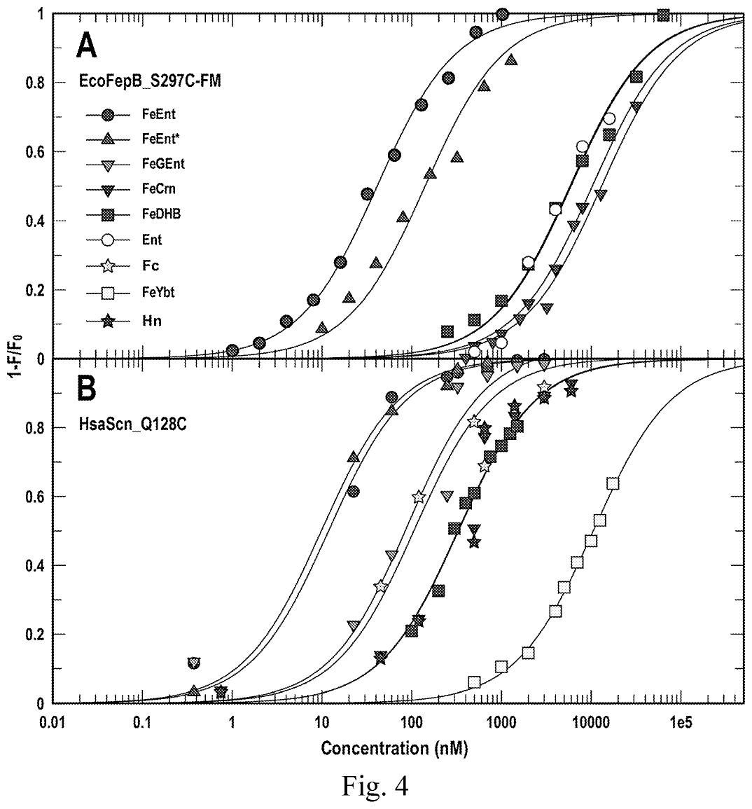

FIG. 4 shows binding of ferric siderophores by EcoFepB and HsaSCN. After expression and purification of EcoFepB_T297C-FM (panel A) and the HsaSCN_Q128C-FM (panel B) we evaluated the affinities of the two binding proteins for a spectrum of apo and ferric siderophores. For each data point we made 3 determinations of quenching in response to the addition of different concentrations of an iron complex. The curves represent non-linear fits of the mean values to a single-site binding model using Grafit 6.0.12. The mean standard errors of the resulting K_{D} values from the fitted curves were: EcoFepB_S297C-FM, 23%; HsaScn_Q128C-FM, 21%.

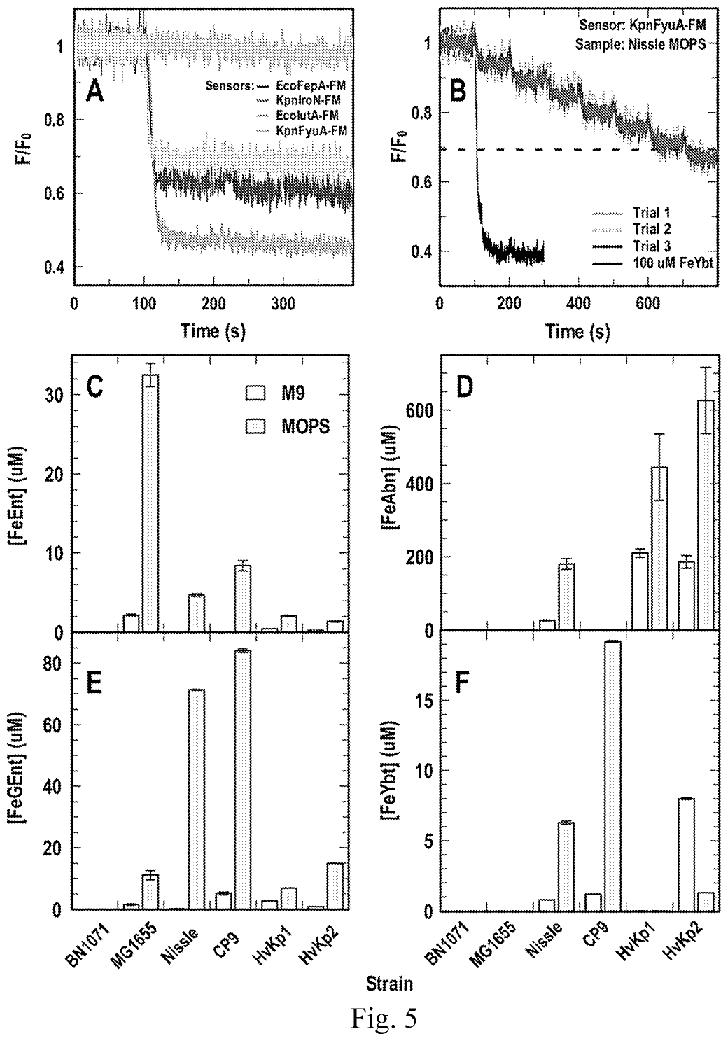

FIG. 5 shows FD sensor assays of siderophores in culture supernatants. After bacterial growth in iron-deficient media, we removed the cells by centrifugation, ferrated the culture supernatants and used four fluoresceinated sensor proteins (EcoFepA, KpnIroN, EcoIutA and KpFyuA) to assay for the presence of FeEnt, FeGEnt, FeAero and FeYbt, respectively, in media from E. coli strains B1071, MG1655, Nissle 1917 and CP9, and K. pneumoniae strains HvKp1 and HvKp2. FIG. 5 panel A specifically shows detection of ferric siderophores. Undiluted culture supernatant, in this case from CP9, immediately revealed the presence or absence of the compounds of interest. The quenching patterns from the 4 FLGP sensors, when an aliquot of supernatant was added at 100 s, showed that CP9 made Ent, GEnt and Ybt, but not Abn. FIG. 5 panel B specifically shows determination of ferric siderophore concentrations in culture supernatants. For several FD sensors (e.g., KpnFyuA, shown here), the quenching from sequential additions of spent ferrated media (in this case, 3 ul aliquots of a 50-fold dilution of the Nissle 1917 supernatant), added at 100 s intervals), revealed the concentration of ferric siderophore. In each such experiment, we made three individual trials, averaged the spectra, and calculated the variance between the trials, which was minimal. At a level of 50% quenching, [FeYbt]=K_{D}: 1 nM, so we calculated [FeYbt] in the original supernatant from the dilution factor. FIG. 5 panels C-F show production of siderophores by E. coli and K. pneumoniae strains in iron-limited M9 and MOPS media. Using the same sequential titration approach, we interrogated the spent media of several bacterial strains. BN1071 (entA) is a control E. coli laboratory strain that does not make any siderophores; MG1655 is a prototypic laboratory E. coli strain, whereas NIssle 1917, CP9 and the K. pneumoniae strains HvKp1 and HvKp2 are wild isolates. Titrations of supernatant aliquots from cultures of these strains divulged the presence and concentrations of Ent (panel C), Abn (D), GEnt (E) and Ybt (F). See also Table 2.

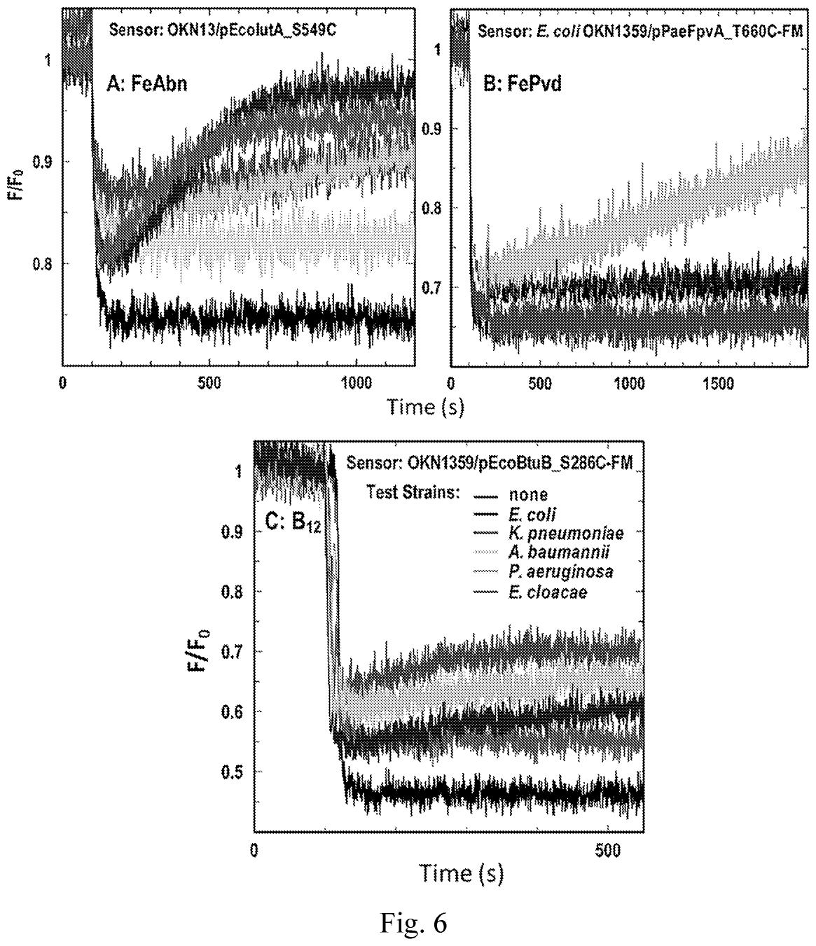

FIG. 6 shows decoy sensor measurements of FeAbn, FePvd and B₁₂ uptake by CRE/ESKAPE pathogens. We expressed three of the FGLP sensors in Table 1 in TonB-deficient E. coli hosts, and incubated the cells at 37° C. with bacteria that were grown in iron-deficient MOPS minimal media: E. coli MG1655 (blue); K. pneumoniae Kp52.145 (red); A. baumannii 17978 (yellow); P. aeruginosa PAO1 (green); E. cloacae (magenta); sensor only (black). The assays observed uptake of FeAbn by all the strains except A. baumannii (panel A), exclusive transport of FePvd by P. aeruginosa (panel B), and uptake of B₁₂ by all the strains except E. cloacae (panel C).

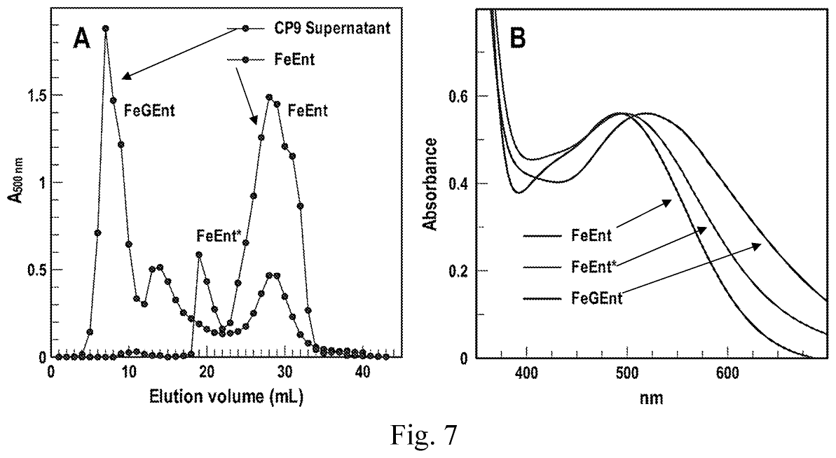

FIG. 7 shows purification and visible spectra of ferric tricatecholate siderophores. FIG. 7 panel A specifically shows a sample of purified FeEnt was stored for 2 weeks on ice and chromatographed on Sephadex LH20 in 5 mM NaHPO4, pH 7. In addition to FeEnt (fractions 23-33), the absorption of the eluent fractions at 495 nm showed the presence of a degradation product (FeEnt*; fractions 19-21). Chromatography of a concentrated, ferrated T-medium supernatant from E. coli CP9 on the same column revealed FeGEnt as the most abundant iron complex (fractions 5-11), but a peak that migrated like FeEnt was also present. FIG. 7 panel B specifically shows the visible spectra of the purified iron complexes of FeEnt, FeEnt* and FeGEnt, each at 100 uM, differentiated them from one another.

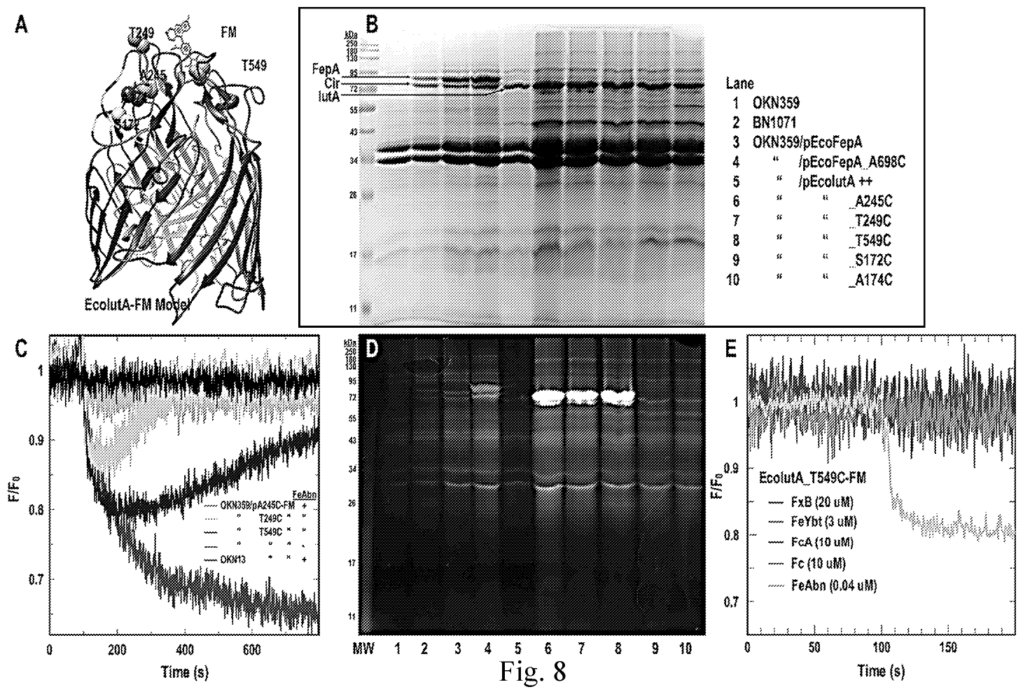

FIG. 8 shows expression, labeling and fluorescence quenching of EcoIutA Cys mutants. FIG. 8 panel A specifically shows a model of EcoIutA, generated by MODELLER of CHIMERA (UCSF), shows the predicted locations of 6 site-directed Cys substitutions that we evaluated as sensors for FeAbn. FIG. 8 panel B specifically shows SDS-PAGE of cell envelope fractions from OKN359 derivatives grown in MOPS media (legend at right) showed mutant protein expression at similar levels to that of EcoFepA (lanes 3 and 4). FIG. 8 panel D specifically shows A fluorescence scan of the same gel revealed high intensity labeling of mutants A245C, T249C and T549C. FIG. 8 panel C specifically shows Among the 3 labeled mutants, EcoIutA_T549C-FM showed the best quenching and TonB-dependent recovery in response to FeAbn (added to 3 μM at t=100 s). FIG. 8 panel E specifically shows EcoIutA_T549C exclusively recognized FeAbn, and did not bind other ferric hydroxamates.

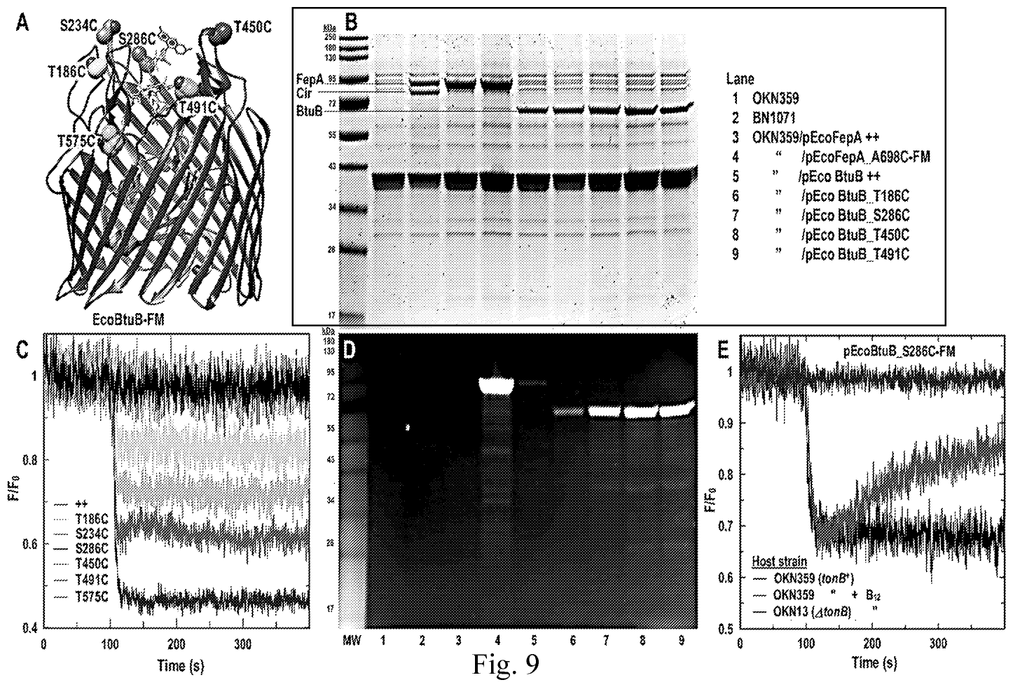

FIG. 9 shows expression, labeling and fluorescence quenching of EcoBtuB Cys mutants. FIG. 9 panel A specifically shows the EcoBtuB crystal structure shows the locations of 6 site directed Cys substitutions that we evaluated as sensors for B12. FIG. 9 panel B specifically shows SDS-PAGE gels of cell envelope fractions from OKN359 derivatives grown in MOPS media showed similar levels of mutant protein expression to that of EcoFepA (lanes 3 and 4; legend at right). FIG. 9 panel D specifically shows a fluorescence scan of the same gel showed high intensity labeling of A244c, T249C and T549C. FIG. 9 panel C specifically shows EcoBtuB_S286C-FM was most strongly quenched by the addition of B12 (10 μM at t=100 s); its uptake reaction was TonB-dependent as shown in panel E.

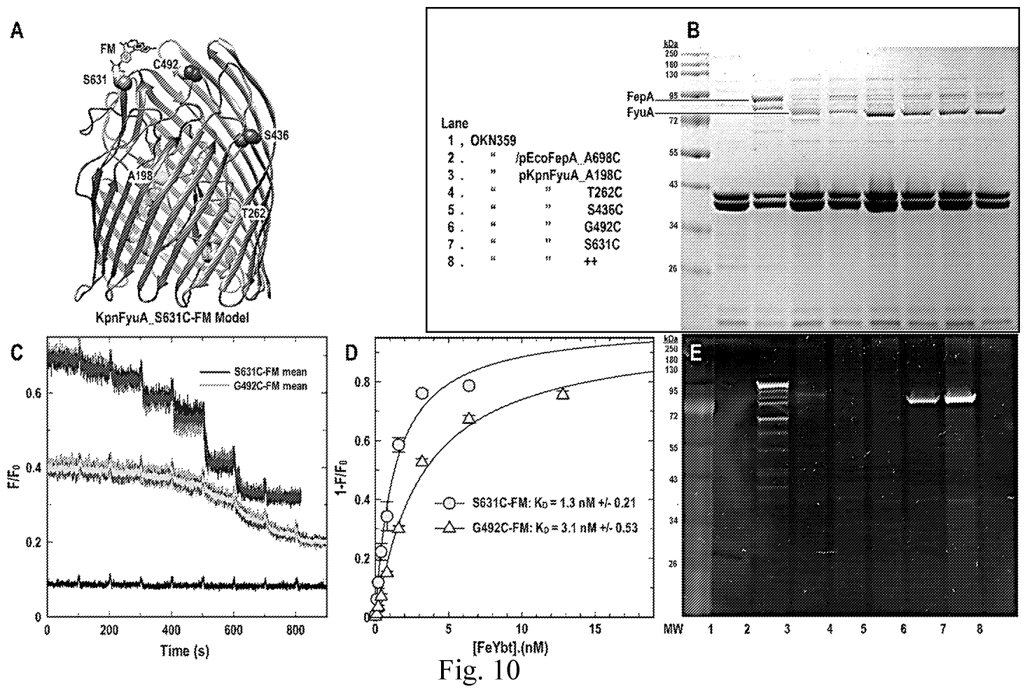

FIG. 10 shows expression, labeling and fluorescence quenching of KpnFyuA Cys mutants. FIG. 10 panel A specifically shows a model of KpnFyuA structure, generated by the MODELLER function of CHIMERA, shows the predicted locations of 5 site-directed Cys substitutions that we evaluated as sensors for FeYbt. FIG. 10 panel B specifically shows SDS-PAGE gels of cell envelope fractions from OKN359 derivatives grown in MOPS media showed mutant protein expression at high levels, similar to that of EcoFepA_A698C-FM (lane 2). FIG. 10 panel E specifically shows fluorescence scan of the same gel revealed acceptable labeling of mutants G492C and S631C. FIG. 10 panel C specifically shows titrations of sensor cells. At 100 s intervals sequentially increasing concentrations of FeYbt were added to OKN359 expressing either KpnFyuA wild type (1 trial; black tracing), mutant G492C-FM (3 trials; green tracings with yellow mean) or mutant S631C-FM (3 trials; blue tracings with red mean). FIG. 10 panel D specifically shows non-linear curve fits of the concentration dependence of the extent of quenching to a single site binding model (Grafit, Erithacus) revealed the affinities of their binding reactions: G492C-FM=3.1 nM; S631C-FM=1.3 nM.

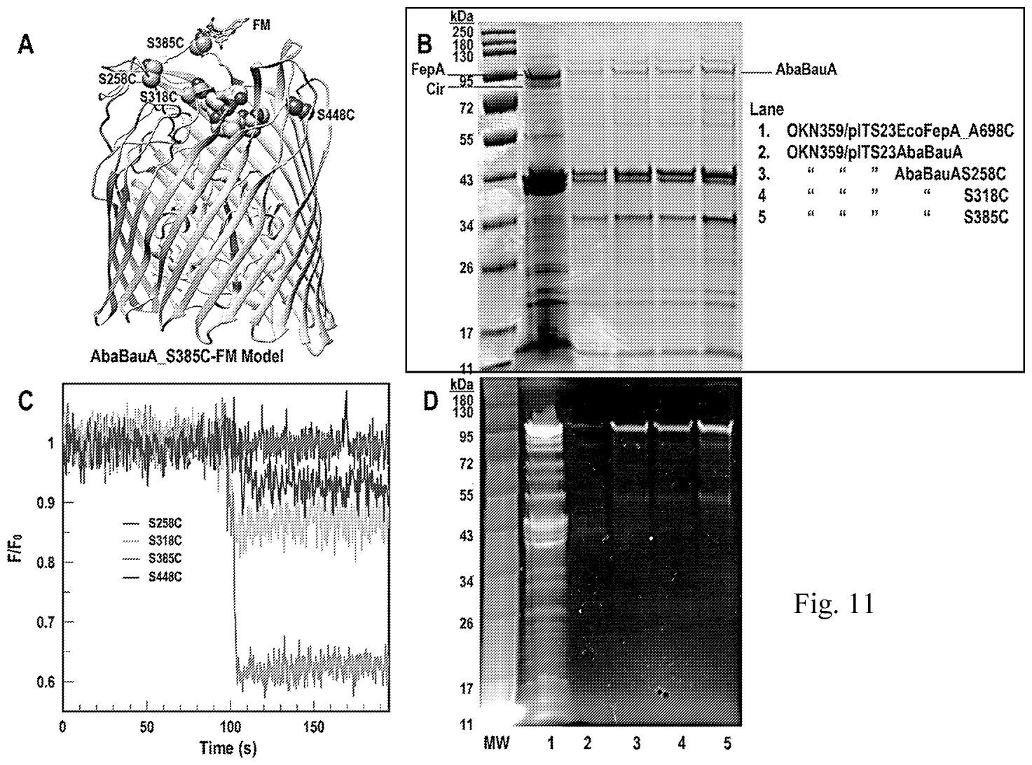

FIG. 11 shows expression, labeling and fluorescence quenching of AbaBauA Cys mutants. FIG. 11 panel A specifically shows the AbaBauA crystal structure shows the predicted locations of 4 site-directed Cys substitutions that we evaluated as sensors for FeAgn. FIG. 11 panel B specifically shows SDS-PAGE gels of cell envelope fractions from OKN359 derivatives grown in MOPS media showed mutant protein expression, at lower levels than that of EcoFepA_A698C-FM (lanes 2-4), but a fluorescence scan of the same gel (panel D) revealed acceptable labeling of mutants S258C, S318C, and S385C. S385C-FM was most sensitive to quenching by FeAgn panel C).

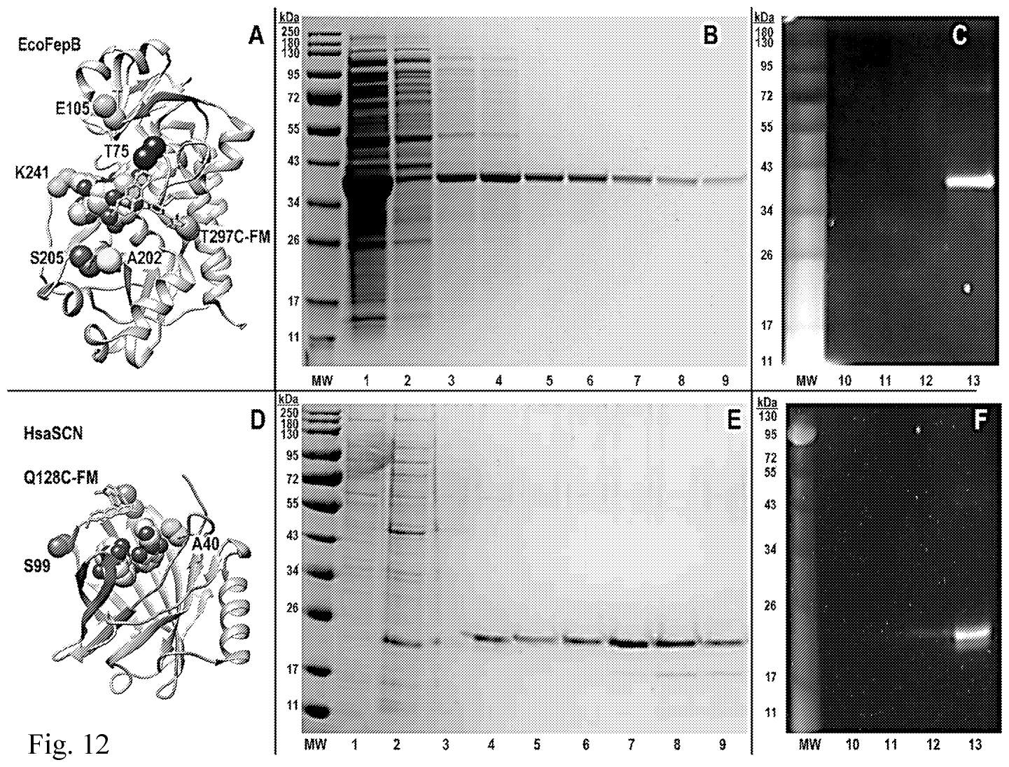

FIG. 12 shows EcoFepB and HsaSen Cys Mutants; purification and FM-labeling. We engineered Cys substitutions in EcoFepB (panel A) and HsaSCN (panel D). Preliminary experiments showed that EcoFepB_T297C and HsaSCN_Q128C were optimal for FM labeling. After growth and IPTG induction of BL21 harboring pEcoFepB T297C-6H or pHsaSCNQ128C-6H, we purified the proteins on Talon Superflow and analyzed the eluates by SDS-PAGE, stained with Coomassie blue (panels B and E, respectively): lane 1, cytoplasm; 2, column pass-through, lanes 3-9 sequential fractions of the 40 mM imidazole eluate. We consolidated the purified proteins, exposed them to 5 μM FM and evaluated the extent of labeling by SDS-PAGE, followed by UV illumination (lane 13 in panels C and F, respectively).

### Discussion of Results

Siderophore-sensor pairs. We engineered FLGP sensors of ferric catecholates (Ent, GEnt, dihydroxybenzoate (DHB), dihydroxybenzoyl serine (DHBS), cefidericol (FDC), MB-1), ferric hydroxamates (Abn, ferrichrome (Fc)), mixed chelators (Ybt, acinetobactin (Acn), anguibactin (Agn), pyoverdine (Pvd)) and porphyrins (hemin (Hn), vitamin B₁₂ (B₁₂)) (Table 1). In our experiments with these compounds, we used the more readily available siderophore anguibactin as a biological equivalent of acinetobactin, because the two chelators are nearly chemically identical and functionally equivalent with regard to recognition by A. baumannii. In total, the LGPs of interest originated from Escherichia coli (EcoFiu, EcoFepA, EcoCir, EcoFhuA, EcoIutA, EcoBtuB), Klebsiella pneumoniae (KpnIroN, KpnFepA1658, KpnFepA4984, KpnFepA2380, KpnFyuA), Acinetobacter baumannii (AbaFepA, AbaPirA, AbaPiuA, AbaBauA), Pseudomonas aeruginosa (PaeFepA, PaeFpvA) and Caulobacter crescentus (CcrHutA). We made additional sensors from EcoFepB, a periplasmic binding protein that acts in ferric catecholate uptake, and human siderocalin (HsaSCN), that recognizes multiple siderophores. We cloned the LGP structural genes in the low-copy plasmid pHSG575, and the binding protein genes in pET28a-β (+), that added a 6-His tag to their N-termini. After verifying the clones by DNA sequencing, we changed a handful of residues in each protein to cysteine. Nearly all of these OM proteins are devoid of cysteine; the few exceptions contain Cys pairs that are unreactive unless subject to reduction by β-mercaptoethanol or other chemical agents. We chose the target residues for site-directed Cys mutagenesis from analysis of crystal structures (EcoFepA, EcoCir, EcoFiu, EcoFhuA, EcoBtuB, KpnFyuA, AbaPiuA, AbaPirA, AbaBauA, HsaSCN), or by their location in hypothetical structures predicted by the Modeller algorithm of CHIMERA (CcrHutA, EcoIutA, KpnFepA, KpnIroN, AbaFepA, PaeFepA). After construction and verification, we expressed the proteins in E. coli, subjected each Cys mutant LGP to modification by fluorescein maleimide (FM; 5 μM for 15 min at 37° C.), and then analyzed their expression, fluoresceination, and fluorescence quenching during binding of a metal complex. Relative to pathogenic host organisms, the production of FLGP in non-pathogenic, rough (rfaB) E. coli K-12 facilitated their optimum FM labeling in a cell surface environment that is unobscured by LPS O-antigen or capsule. We obtained high level, usually stoichiometric modification of the engineered Cys sulfhydryls with extrinsic maleimide fluorophores (FIGS. 8-12). Certain Cys locations were better or worse for some LGP (e.g., AbaBauA, EcoFiu), but we always found accessible labeling sites in each OM protein. We then chose the most sensitive single mutant of each LGP, and spectroscopically determined its specificities and affinities for the apo- and ferric siderophores in relevant chemical classes (Table 1). For production of HsaSCN and EcoFepB, we introduced Cys substitutions at positions near their binding sites, over-expressed the proteins and purified them from cell lysates by metal ion chromatography (FIG. 12). For each LGP or soluble binding protein sensor, we studied three or more Cys substitutions to identify an effective site for attachment of an extrinsic fluorophore. To optimize detection and quantification of the individual metal complexes, we compared the fluorescence intensities of the different Cys mutant derivatives, as well as the extents of their quenching by their homologous and heterologous ligands.

LGP binding promiscuity. The high (nanomolar) affinity of certain LGP for a particular ligand suggested potential exclusivity in their binding reactions. EcoFepA, for example, tightly adsorbs FeEnt (K_{D}=0.1-0.4 nM), which allows it to scavenge sub-nanomolar concentrations of FeEnt. However, we found that EcoFepA does not have exclusive specificity for FeEnt. Like PaeFpvA, the EcoFepA-FM sensor had considerable affinity for the aposiderophore, Ent (K_{D}=43 nM). As we surveyed other FLGP in our panel this result became a general trend: 11 other FLGP showed ˜100-fold lower binding affinity for their corresponding aposiderophores. Thus, FeEnt was the primary ligand of EcoFepA, KpnFepA1658 and 4984, AbaFepA, PaeFepA, KpnIroN and EcoFepB, but they all also adsorbed Ent with 70-200-fold lower affinity (Table 1). Receptors for non-phenolate iron complexes showed the same duality: EcoFhuA, EcoIutA and KpnFyuA bound apoFc, Abn and Ybt with 60-200-fold lower affinity than seen for their ferric complexes. In addition to aposiderophores, EcoFepA also recognized other chemically similar iron complexes. Several different forms of catecholate chelators are relevant to Gram (−) bacterial iron acquisition. Besides glucosylation, the three catecholate groups of FeEnt may oxidize to quinones, and the lactone ring that conjoins them may hydrolyze to yield linear tri-, di- and monocatecholates. These degradation products (FeEnt*) arose even when FeEnt was stored on ice. Although we typically removed them by chromatography on Sephadex LH20, we also studied their interactions with the FLGP. EcoFiu and EcoCir were previously linked to the uptake of ferric monocatecholates, but unexpectedly, FD assays showed that EcoFepA-FM adsorbed FeEnt* (K_{D}=8 nM), and had weak affinity for FeGEnt (K_{D}=380 nM) and FeDHBS (K_{D}=2000 nM). This was another trend: besides highest affinity for a primary ligand (EcoFepA: FeEnt; EcoFiu: FeEnt*; EcoCir: FeEnt*, EcoFhuA: Fc), many LGP sensors manifested secondary, lower affinities for structurally related metal chelates (FIG. 2, Table 1).

The specificities of orthologous LGP in related organisms were unpredictable and complicated, as illustrated by the ferric catecholate uptake systems of Gram (−) ESKAPE pathogens. First, the EcoFepA orthologs KpnFepA1658 (82% identity) and PaePfeA (61% identity) had the same ligand preferences as EcoFepA (FeEnt>FeEnt*>FeGEnt). However, AbaFepA (46% identity) differed, in that it bound FeEnt, FeEnt* and FeGEnt with about the same affinity (K_{D}≈25 nM). Secondly, other orthologous ferric catecholate receptors displayed both recognition correspondences and differences. EcoCir and AbaPirA were a pair (37% identity), with considerably lower (micromolar) affinities for iron complexes. Both bound FeEnt* (K_{D} values of 7 and 1 μM, respectively), but both also recognized other iron complexes, with the preferences (Table 1):
EcoCir: FeFDC>FeEnt*≈FeDHB>Fe-MB-1>FeEnt;
AbaPirA: FeEnt*>FeFDC>FeEnt>FeMB-1>FeDHB.
   Similarly, EcoFiu and AbaPiuA (31% identity) both manifested lower (generally micromolar) binding affinities for ferric catecholates (Table 1), that produced different priorities:
EcoFiu: FeDHBS>FeGEnt>FeEnt*>FeEnt≈FeFDC:
AbaPiuA: FeEnt*>FeEnt>FeDHB>FeFDC>FeMB-1.

EcoFiu did not bind FeDHB; AbaPiuA did not bind FeGEnt nor FeDHBS. So, in both cases, despite their annotation as orthologs, EcoCir and AbaPirA, and EcoFiu and AbaPiu were quite different in their binding specificities.

Lastly, KpnIroN grouped with EcoFiu (21% identity), EcoFepA (51% identity) and AbaFepA (49.4% identity), in that all four proteins recognized FeEnt, FeEnt* and FeGEnt, but with different priorities:
EcoFepA: FeEnt>FeEnt*>FeGEnt;
EcoFiu: FeGEnt>FeEnt*>FeEnt;
KpnIroN: FeEnt>FeGEnt>FeEnt*;
AbaFepA: FeGEnt≈FeEnt*≈FeEnt.
   All the FepA orthologs also recognized some combination of ferric monocatecholates, with lower affinity.

Besides their divergent specificities, some LGP in the catecholate receptor group had much lower binding and transport affinities for their ligands. Overall, the ferric catecholate receptors spanned a 10,000-fold range of affinity, from sub-nanomolar to 10-15 μM K_{D} values, suggesting that certain receptors (AbaPiuA, EcoCir, AbaPirA, AbaBauA) are only functionally relevant when their ferric siderophore ligands are present at comparatively high (i.e., μM) concentrations (FIG. 2, Table 1). Overall, the catecholate-specific FLGP revealed that Gram (−) bacteria produce assortments of OM proteins that discriminate among the ferric siderophores they encounter in nature or in the host environment. The different selectivities and affinities of these receptors allows the bacteria to adapt to a variety of siderophore compositions and concentrations.

LGP binding selectivity. Although the ferric catecholate LGP sensors displayed broad recognition specificity (FIG. 2), other LGP sensors showed singularity for one metal complex (FIG. 3). EcoIutA, EcoBtuB, CcrHutA, KpnFyuA, AbaBauA and PaeFpvA were nearly or fully exclusive in their binding of FeAbn, B₁₂, hemin, FeYbt, FeAgn and FePvd, respectively. Among those, only KpnFyuA (that weakly bound FxB) and PaeFpvA (that weakly bound FeEnt, FeEnt* and FeDHB) had any affinity for other ferric siderophores besides their primary ligands. IutA, for example, had high affinity for FeAbn (K_{D}=6 nM), and did not measurably bind any other ferric hydroxamates (Fc, FcA, FxB). Each of these more specific receptors was aptly suited to its biochemical role. Abn biosynthesis and FeAbn uptake are often encoded together on transferable plasmids; bacteria that harbor such plasmids express a complete system for siderophore production and ferric siderophore acquisition, that confers invasiveness in the host. Hence, EcoIutA's selectivity for FeAbn, and its blindness to other ferric complexes, well complement Abn production by virulent strains. Likewise, KpnFyuA and AbaBauA exclusively bound ferric siderophores associated with the pathogenicity of their producers. CcrHutA and EcoBtuB were also specific for iron and cobalt porphyrins, respectively (FIG. 3).

Specificities of EcoFepB and HsaSCN. The periplasmic protein FepB recognizes and binds FeEnt after its internalization through the OM, and the innate immune component SCN opposes the iron scavenging of bacterial pathogens by adsorbing apo- and ferric siderophores, thereby reducing their concentrations in tissues, body fluids, blood and serum. After cloning EcofepB and the cDNA of the human gene HsaSCN in pET28, we genetically engineered the substitutions T297C and Q128C, respectively, purified the over-expressed his-tagged mutant binding proteins by metal affinity chromatography, and labeled them with FM. As expected, the periplasmic binding protein EcoFepB-FM preferentially recognized FeEnt (K_{D}=43 μM), about the same as reported for the native protein, and with affinity that was about 100-fold less than EcoFepA. EcoFepB also bound Ent, FeEnt*, FeGEnt, FeCrn and FeDHB, with a range of affinities. In that sense, it resembled HsaSCN. The serum FBP sensor preferred FeEnt and FeEnt* (K_{D}=10-12 nM) among the apo and ferric siderophores we tested. HsaSCN-FM also showed sub-micromolar affinity for other ferric catecholates, including FeGEnt (K_{D}=0.09 UM), FeDHB (K_{D}=0.33 UM) and FeCrn (K_{D}=0.33 μM). It similarly adsorbed ferric hydroxamates and mixed ferric chelates, usually with sub-micromolar affinity: Fc (K_{D}=0.11 μM), FxB (K_{D}=0.18 μM), FeAgn (K_{D}=0.16 μM), FeYbt (K_{D}=11 μM). Overall, HsaSCN adsorbed many iron chelates, more than any of the LGP, with affinities over 3 logs of concentration (10 nM-11 μM; FIG. 4).

Measurement of siderophores in complex samples. FD sensors that possess high affinity for a single ligand can unambiguously detect, identify and quantify that compound in experimental samples. To illustrate this capability, we grew laboratory (MG1655), probiotic (Nissle 1917) and pathogenic (CP9) strains of E. coli, and two hypervirulent strains of K. pneumoniae (HvKp1, HvKp2) in iron deficient MOPS and M9 minimal media, removed the cells by centrifugation and analyzed the supernatants with FD sensors. Under these conditions the bacteria maximize siderophore production, in some cases so much that ferration of the spent media resulted in deeply red- or orange-colored solutions. The genomic and biosynthetic pathways of these strains implied the production of Ent, GEnt, Abn and Ybt. Therefore, we interrogated their ferrated supernatants with EcoFepA-FM, EcoIutA-FM, KpnIroN-FM and KpnFyuA-FM (FIG. 5; Table 2). The sensors found only 3 of the 4 siderophores in the CP9 and HvKpn1 supernatants, and they were different between the two: in the former case, FeEnt, FeGEnt, and FeYbt; in the latter case, FeEnt, FeGEnt and FeAbn. HvKpn2, on the other hand, produced all 4 siderophores, which enhances its potential virulence relative to the other strains.

The detection of non-catecholate ferric siderophores (FeAbn, FeYbt) was relatively straightforward because their FLGP (EcoIutA-FM and KpnFyuA-FM, respectively) were virtually monospecific and unimpaired by interference from other ferric complexes (Table 1, FIG. 3). On the other hand, the quantification of ferric catecholates was complex, because the best sensors for FeEnt (EcoFepA-FM) and FeGEnt (KpnIroN-FM) also bound (albeit with lower affinity) other catecholate iron chelates (Table 1, FIG. 2). This dual recognition of FeEnt and FeGEnt by EcoFepA-FM and KpnIroN-FM complicated the quantification of GEnt. However, because of its ˜600-fold higher affinity for FeEnt than for FeGEnt (Table 1), EcoFepA-FM accurately measured [FeEnt], even if FeGEnt was present at >100-fold higher levels (Table 2, FIG. 4). Once the [FeEnt] in a solution was known, the extent of quenching of KpnIroN (which binds FeGEnt; K_{D}=7 nM) by the same solution provided information about the presence of FeGEnt (Experimental Procedures).

These sensor measurements revealed other things about the strains and their growth in the two media. First, all strains grew more in MOPS media, achieving cell densities of 3-5×10⁹/mL, relative to 0.5-1×10⁹/mL in M9 (Table 2). Siderophore biosynthesis was correspondingly greater in MOPS, usually 5-10-fold higher, or more, than in M9 (Table 2, FIG. 5). Second, despite these general trends, the strains were individually unique with regard to siderophore production in iron-deficient conditions. MG1655 only produced Ent, whereas the wild isolates Nissle, CP9, HvKp1 and HvKp2 produced combinations of the siderophores that we assayed: Nissle and HvKp2 produced all four, and as noted above, CP9 did not make Abn and HvKp1 did not make Ybt (FIG. 5). Third, after 24 h growth in iron-deficient MOPS media, the total siderophore concentrations approached millimolar levels. This abundant growth and copious siderophore production illustrated the suitability of MOPS minimal medium for imposition of iron deficiency. Lastly, correlations between siderophore biosynthesis and bacterial virulence were discernable, but also cryptic. The ExPEC strain CP9 excreted a profuse amount of GEnt, but no Abn. Conversely, the defining characteristic of both hypervirulent K. pneumoniae strains was massive secretion of Abn, ˜50-fold more than their level of GEnt production.

Measurements of metal transport. Besides their recognition and discrimination of metal complexes, FD sensors monitored the uptake of their ligands by cells of interest (FIG. 6). In AtonB hosts LGP can bind but cannot transport their metal complexes. When FeEnt binds to OKN13 (AtonB)/pEcoFepA-FM it quenches fluorescence, but the strain's TonB-deficiency prevents FeEnt uptake. As a result, the fluorescence of EcoFepA-FM inversely relate to [FeEnt] in its environment. When such FD sensor cells cohabit an environment with transport-active bacteria they reveal their ligand's concentration, and therefore also its uptake by the cohabitant. EcoFepA avidly binds FeEnt (K_{D}=0.1-0.4 nM); 40 nM FeEnt quenches EcoFepA-FM ˜99%. Such high-affinity receptors, with nanomolar K_{D} values, are most effective decoy sensors, because they rapidly detect the transport (depletion) of nanomolar amounts of ferric siderophores or porphyrins by the bacteria. Iron deficient Gram (−) bacteria, with maximally expressed, chromosomal FepA, transport FeEnt at ˜50 pMol/10⁹ cells/min, so in FLGP assays with ˜2.5×10⁷ cells/mL in a cuvette, such strains deplete 40 nM FeEnt in <40 min. Lower affinity FLGP with higher K_{D} values (e.g., AbaFepA, K_{D}=17 nM (Table 1)) require higher concentrations of ligand (1.7 μM FeEnt) to comparably quench the sensor. Consequently, it takes longer for test strains to deplete this 100-fold higher concentration of FeEnt, and assays with an AbaFepA-FM sensor show slower fluorescence recovery.

Besides EcoFepA-FM-FeEnt, we tested the abilities of three other decoy sensor-ligand pairs (EcoIutA-FM-FeAbn; PaeFpvA-FM-FePvd; EcoBtuB-FM-B₁₂) (FIG. 6) to monitor metal uptake by Gram (−) CRE/ESKAPE bacteria. The sensors revealed complex patterns of utilization. Almost all of the strains acquired FeAbn (except A. baumannii), but at different rates: iron-restricted E. coli and E. cloacae transported FeAbn roughly 2-fold faster than P. aeruginosa or K. pneumoniae. Similarly, all the test strains but one (E. cloacae) transported B₁₂, but at a slower rate than the uptake of ferric siderophores by the test strains. Lastly, only P. aeruginosa transported FePvd, and it did so at a considerably slower rate than E. coli transported FeEnt (53) or FeAbn. Thus, the FLGP quantitatively defined the transport of the noted compounds by the test strains, demonstrating the generality of the decoy sensor approach to uptake measurements.

### Discussion

The conversion of binding proteins into FD sensors is an evolution of Cys scanning mutagenesis, that that has been previously used to comprehensively analyze and manipulate the membrane transporter LacY. It has been shown that neither the elimination of native Cys residues nor the introduction and chemical modification of genetically engineered Cys side chains were strongly detrimental to the function of the lactose permease. Similar research on EcoFepA and other LGP reiterated the benefits of site-directed chemical modifications of OM proteins. One of the noteworthy attributes of FLGP and FBP sensors was their facile determination of the breadth of their own specificity. The relative affinities of their binding reactions defined each receptor's ligand preference. In a broader sense, these data reveal which nutrients, vitamins and metals a bacterium acquires from its environment. The orthologs and paralogs of EcoFepA illustrated the insight that FLGP sensors provide. Despite its annotation, AbaFepA had little similarity to EcoFepA in regard to its affinities and specificities for catecholate iron complexes. EcoFepA, KpnFepA1658 and PaeFepA, on the other hand, were consistent in their primary affinity for FeEnt. EcoFiu and EcoCir were previously implicated in the uptake of monocatecholates, but besides compounds like FeDHBS, FeFDC and FeMB-1 they bound FeEnt, FeEnt*, and FeGEnt (EcoFiu only). These findings highlighted a general promiscuity in the catecholate receptors, and showed the utility of FLGP in unraveling the complexities of their own specificities. Additionally, the tactic of site-directed fluorescence modification of receptors and transporters creates a biochemical path to the verification of genomic sequence annotations.

The sensor assays described hierarchies of receptor preferences for different forms of catecholates compounds, including the siderophore antibiotics MB-1 and cefiderocol. Such studies are valuable in early phase discovery of new antibacterial drugs. A candidate compound's MIC against target pathogens depends on its accumulation in the correct bacterial compartment (the periplasm for β-lactams), which is difficult to measure. The influx rate for siderophore-antibacterials depends on TonB-dependent intake through one or more LGP. Without knowing which LGP are involved, it is a blind empirical discovery process, that has led to the costly failure of many siderophore-β-lactams, and the success of only one compound, cefiderocol. Information from FLGP sensors will inform and de-risk the drug discovery process, by discerning the spectrum of pathogens (or limitations) for candidate compounds, as well as risks from “adaptation-based” or mutational resistance. It is problematical that the identities and affinities of the LGP involved in cefiderocol uptake are still unknown in all the species for which it is approved for clinical use. Just as it is important to know the target of an antibacterial drug, it is also important to know the route of its entry into each pathogen. In this sense, it was of interest that the first licensed Trojan Horse antibiotic, cefidericol (FeTroja, Shionogi Inc.), was recognized by several FLGP. Its clinical efficacy was already demonstrated against A. baumannii, which concurs with the observation that besides EcoFiu, EcoCir and KpnIroN, it bound to AbaPiuA, AbaFepA, and AbaPirA.

The extension of this technology to the analysis of clinical samples from human or animal hosts may reveal physiological functions that confer pathogenicity or infectivity. The production of particular siderophores connects to the colonization, tropism, invasiveness or lethality of particular bacteria. Some microbes produce virulence-enhancing siderophores, as illustrated by A. baumannii's synthesis of acinetobactin, baumoferrins and fimsbactins. At present, the utilization of iron complexes of these three siderophores appears species-specific, and unique to the pathogenicity of A. baumannii. Other siderophores, or groups of siderophores, more broadly correlate with virulence. The association of Abn with invasiveness was the first example of this relationship, but the simultaneous elaboration of Abn, Ybt and GEnt by isolates of E. coli, K. pneumoniae, S. typhimurium and other bacteria reaffirmed this phenomenon. Each of the siderophores in this mixture promotes the infection of specific organs, increasing the virulence of organisms like hypervirulent K. pneumoniae. Certain siderophores also affect the overall outcome of encounters between bacteria and humans, by influencing the fine line between colonization and pathogenesis. The production of the Ent and utilization of FeEnt facilitates colonization of the mammalian gut, whereas glucosylation of Ent to form GEnt, and utilization of FeGEnt, promotes pathogenesis. The distinction between the naked or glucosylated siderophore is that SCN adsorbs Ent/FeEnt and removes them from circulation, but its lower affinity for GEnt/FeGEnt means that it does not similarly eliminate them from fluids and tissues. This evasion of SCN allows the accumulation of potentially nutritive amounts of GEnt in blood, serum and lymph, that may enhance bacterial proliferation. HsaSCN-FM bound a spectrum of apo and ferric siderophores, which concurs with its innate immune function against pathogenic microbes. Nevertheless, besides GEnt/FeGEnt, neither Abn/FeAbn, nor Ybt/FeYbt, nor Agn/FeAgn adsorbed to HsaSCN: these siderophores consistently link to Gram (−) bacterial pathogenicity. The binding specificities of HsaSCN were already largely known, but the FBP sensor determinations ranked its ligands in the overall hierarchy of its biological activity.

Siderophore production contributes to the infectivity and tissue tropism of K. pneumoniae, and we found that hypervirulent K. pneumoniae (HvKpn2) made significant amounts of Ent, GEnt, Ybt and Abn. However, its massive production of Abn (0.6 mM in MOPS) was ˜40-fold more than that any of the other siderophores, and ˜97% of the total amount of secreted siderophores, supporting the known role of aerobactin in promoting bacterial invasiveness and pathogenicity. To the contrary, E. coli Nissle 1917, a non-pathogenic probiotic strain, made the same four siderophores in comparable, but different amounts. It also produced more aerobactin (0.2 mM) than any other siderophore. Hence, elaboration of siderophores is not a stand-alone virulence determinant of the Enterobacteriaceae. Other attributes of K. pneumoniae (as for example, its hypermucoviscosity), synergize with siderophore biosynthetic and transport systems to maximize the infectivity of certain pathogenic strains.

### Experimental Procedures

Bacteria and plasmids. E. coli laboratory strains descended from BN1071 (entA; OKN1 (ΔtonB), OKN3 (ΔfepA), OKN7 (ΔfhuA), OKN13 (ΔtonB, ΔfepA) and MG1655. E. coli strains Nissle 1917 and CP9 are probiotic and ExPEC wild isolates, respectively. ESKAPE strains and other wild isolates included K. pneumoniae Kp52.145 (courtesy of Regis Tournebiz, Institut Pasteur), A. baumannii ATCC 17978, P. aeruginosa PA01 (courtesy of Stephen Lory, Harvard University). K. pneumoniae Kp52.145 was the source of four annotated kpnfepA structural genes: chromosomal loci 1658, 2380, 4984, and plasmid (pII) locus 0027. We obtained the HsaSCN structural gene in plasmid GST-h SCN_pGEX-4t-1 from Roland K. Strong.

Media, siderophores and other metal complexes. We grew bacteria in LB and subcultured at 0.5-1% into iron-deficient MOPS or M9 minimal media for 6-24 h to achieve iron limitation. We prepared apo and iron complexes from a collection of ˜40 purified catecholate, hydroxamate and mixed chelation siderophores, or purified the compounds of interest from bacterial culture supernatants (FIG. 7). In each case, we measured and adjusted their concentrations from their UV-visible absorption spectra. For determinations with acinetobactin (Acn), we substituted anguibactin (Agn). The two mixed chelation siderophores are nearly chemically identical, but the iron complex of the latter is more readily available, and known to bind to the BauA receptor of A. baumannii. Other compounds were from commercial sources (DHBS, KeyOrganics; cefiderocol, Shionogi.; MB-1, Venatorx).

Selection of Cys substitution mutant targets. For each LGP of interest we chose multiple candidate residues, located in the surface loops of their outer vestibules, for Cys substitution and fluorescent modification. In most cases, crystal structures guided the selections EcoFepA (1FEP), EcoBtuB (1NQF), EcoFhuA (1BY5), EcoFiu (6BPM), EcoCir (2HDF), AbaBauA (6H7F), AbaPirA (5FR8), AbaPiuA (5FP1), YpeFyuA (4EPA). In other cases (CcrHutA, EcoIutA, KpnFepA, KpnIroN, PaeFepA) we used CLUSTALQ to identify the closest structurally solved phylogenetic ortholog or paralog of the unsolved LGP, on the basis of percent sequence identity, and then predicted its structure with the MODELLER function of CHIMERA (UCSF). After ensuing construction of multiple mutants for each receptor protein, we evaluated their accessibility to chemical modification and their sensitivity to fluorescence quenching to determine the best construct for each of the siderophores under investigation (FIGS. 8-12).

Site-directed Cys mutagenesis. As a general approach, we used PCR to clone the LGP of interest (Table 1) from the chromosomes of selected bacterial species. Because we ultimately expressed all the LGP-derived sensors in the E. coli OM, we precisely cloned the nucleotide sequences encoding their mature proteins and inserted them downstream from the native promoter and signal peptide of EcofepA in pITS23, a derivative of the low-copy pHSG575. We used QuikChange (Stratagene) for single Cys substitutions in the LGP of interest, with complementary oligonucleotides flanking the mutation, followed by digestion of the wild-type vector by DpnI. We confirmed the mutations by sequencing (Genewiz) of purified plasmids.

Cys Mutant protein expression and fluorescent labeling. The vector for LGP production, pITS23, carries wild-type EcofepA under control of its native, Fur-regulated promoter. For expression of each of the E. coli LGP (Fiu, FhuA, IutA, Cir, BtuB) we replaced EcofepA in pITS23 with the alternate LGP structural gene, with its own signal sequence, such that the iron-regulated EcofepA promoter-controlled biosynthesis. For LGP from of other species (KpnFepA, KpnIroN, KpnFyuA, AbaBauA, PaeFepA, PaeFpvA, CcrHutA), we replaced the sequence encoding mature EcoFepA with the sequence encoding the foreign mature LGP, downstream from the EcoFepA signal sequence, and regulated by the EcofepA promoter. This strategy allowed the EcoFepA signal sequence to direct secretion and assembly of the foreign OM protein in the E. coli OM. We transformed the constructs into entA, tonB⁺ (OKN3, OKN359) or entA, ΔtonB (OKN13, OKN1359) E. coli hosts, such that growth in iron-deficient MOPS media caused overexpression of the sensor proteins. We fluoresceinated the LGP of E. coli/pITS23 constructs in situ, in living cells. For the soluble binding proteins, we purified the 6His-tagged Cys mutant proteins from cell lysates by metal affinity chromatography, modified them with 5 μM FM at pH 6.75 for 15 min, and re-purified the fluorescently labeled sensor by gel filtration chromatography. We evaluated the expression and labeling of each of the cloned LGP by growing the appropriate E. coli host strain (OKN3, OKN359, OKN13 or OKN1359), harboring the plasmid constructs of interest, in iron-deficient MOPS medium to late log phase (A_{600 nm}=2.5-3.5). We subsequently analyzed SDS-PAGE resolved samples of bacterial OM, or purified protein fractions, for fluorescence emissions at 520 nM before staining with Coomassie blue R (FIGS. 7, 8, 9). In some cases, we used mouse monoclonal (α-EcoFepA) antibodies or rabbit polyclonal (α-FepB) antisera to determine the expression levels and stoichiometry of FM-labeling, which allowed quantification of expression. In each case we obtained comparable, but sometimes less, iron-regulated expression of the heterologous LGP under control of the EcofepA promoter.

Siderophore nutrition tests. To qualitatively evaluate the FeEnt uptake abilities encoded by the mutant fepA alleles, we performed siderophore nutrition tests. We expressed the results as the diameter of bacterial growth around the paper disk, and compared mutant halos to the growth halo conferred by a strain harboring pITS23 (fepA+). These experiments confirmed the transport functionality of the cloned proteins.

Preparation of cell envelope fractions. After growth in LB broth overnight, we sub-cultured FepA mutant derivatives on pITS23 in OKN3, OKN13, OKN359 or OKN1359 at (1%) into 20 mL of iron-deficient MOPS media and incubated the cultures with shaking for 5.5-6 h at 37° C., to an A600 nm=1-1.2 (5-6×10⁸ cells/mL). In some cases, we subjected the cells to fluorescence modification at this stage, as discussed below. After washing with 50 mM NaHPO4, pH 6.7, we collected the cells by centrifugation at 7,500×g, re-suspended the pellets in 2 mL of PBS containing trace amounts of DNase and RNase, and passed the cell suspensions (3×) through a French pressure cell at 14,000 psi. After spinning the lysates at 3000×g for 10 min to remove unbroken cells and debris, we transferred the supernatants to microcentrifuge tubes and pelleted the cell envelopes by centrifugation at 13,000×g for 45 min. We re-suspended the cell envelopes in 400 μL of 50 mM Tris-Cl, pH 7.4, and analyzed expression and the extent of fluorescence labeling by SDS-PAGE and UV illumination.

Fluorescence modifications. For modifications with maleimide fluorophores, we inoculated E. coli strains OKN3, OKN13, OKN359 or OKN1359, harboring plasmids that encoded a Cys mutant LGP, from frozen stocks into LB, grew them overnight and sub-cultured at 1% into iron-deficient MOPS minimal media, with shaking (200 rpm) at 37° C. for 10-12 h, until late exponential phase. After collecting the cells by centrifugation at 7500×g for 15 min, and washing with 50 mM NaHPO4, pH 6.7, we labeled the Cys-mutant LGP with 5 μM FM in 50 mM NaHPO4, pH 6.7 for 15 min at 37° C. FM. We terminated the labeling reactions with 100 μM β-mercaptoethanol. After collecting the fluoresceinated cells by centrifugation at 7500×g for 15 min, and washing with 50 mM NaHPO4, pH 6.7, we re-suspended them in PBS plus 0.2% glucose. We either immediately used the labeled cells in spectroscopic experiments, or rested them on ice (up to 24 h), or added glycerol to 15% and stored them (indefinitely) as 1 mL aliquots at −70° C. In the latter case, after thawing the labeled cells we pelleted them by centrifugation in a microfuge, washed them once with and re-suspended them in PBS containing 0.2% glucose. Lastly, for evaluation of protein expression or the extent of FM-labeling we solubilized aliquots of the cell suspensions with sample buffer, and subjected them to SDS-PAGE. After electrophoresis, we first visualized the fluorescence labeling on a Typhoon 8600 Biomolecular Scanner (GE/Amersham), and then stained the gels with Coomassie blue R (153).

SDS-PAGE and Western Immunoblots. We electrophoretically analyzed cell envelope fractions of the mutants. For SDS-PAGE (154,155) we suspended 30 μg of cell envelope protein (calculated from absorbance at 280 nm) in sample buffer containing 1% SDS and 3% BME, boiled the sample for 5 min, and resolved the samples by on 11% acrylamide/0.3% bis acrylamide slabs at 30 mA. To enhance the separation of proteins in the 80 kDa range, we continued electrophoresis at 30 mA for an additional 45 min after the tracking dye left the gels. [¹²⁵I]-protein A immunoblots provided precise data on the concentration of EcoFepA, and the stained SDS-PAGE gels revealed the concentrations of the other LGP in the OM, relative to EcoFepA.

Fluorescence spectroscopic binding determinations. We observed fluorophore-labeled cells in an SLM AMINCO 8100 fluorescence spectrometer, upgraded with an OLIS operating system and software (OLIS SpectralWorks, OLIS Inc., Bogart, GA), to control its shutters, polarizers and data collection. We also utilized an OLIS Clarity fluorescence spectrometer for fluorescence assays. For binding determinations we deposited 2.5×10⁷ labeled cells in a quartz cuvette (final volume, 2 mL) with stirring at 37° C., measured the initial fluorescence (F₀), and then added increasing concentrations of an iron complex while monitoring the quenching of fluorescence emissions (F) at 520 nm. We performed each measurement in triplicate and calculated the mean value of F/F₀ at each ligand concentration. We plotted 1−F/F₀ vs [ligand] and analyzed the data by the 1-site binding model of Grafit 6.0.12 (Erithacus Ltd. West Sussex, UK), that fits data to a single site saturation curve, where the amount of ligand bound is plotted as a function of the amount free:

$$[ Boun d ] = \frac{C apacity [ Free ]}{{K}_{D} + [ F r e e ]}$$

These plots yielded K_{D} values for each of the receptor-ligand interactions, with associated standard errors.

Measurement of siderophore concentrations in spent media. For analysis of culture supernatants, we inoculated 10-25 mL volumes of iron-deficient M9 or MOPS minimal media with strains of E. coli or K. pneumoniae, shook the cultures at 37° C. for 24 h, removed the cells by centrifugation at 7000×g for 15 min, and added FeCl₃ to 1 mM. After incubating the ferrated supernatants for at least an hour at room temperature, we stored them on ice until they were analyzed.

For concentration measurements using FLGP sensors, we first expressed and fluoresceinated the appropriate LGP, expressed in either OKN13 (ΔtonB, ΔfepA) or OKN1359 (ΔtonB, ΔfepA, Δcir, Δfiu). We next ascertained the initial fluorescence intensity of 2.5×10⁷ sensor cells in 2 mL of PBS in a quartz cuvette (λₑₓ=492 nm; λₑₘ=520 nm), and their maximal quenching by excess ligand. To measure the supernatant concentrations of FeEnt (with EcoFepA-FM), FeAbn (EcoIutA-FM) and FeYbt (KpnFyuA-FM), we removed any precipitated or aggregated material by microcentrifugation at 13,000 rpm for 2 min, and added sequential aliquots of the clarified, ferrated supernatant to the sensor cell suspension, until 50% quenching occurred (i.e., [ferric siderophore]=K_{D}). The dilution factors to 50% quenching divulged the [siderophore] in the original culture supernatant.

Because Ent is the precursor of GEnt, both catecholates are usually present in the media of GEnt producers (FIG. 5). This fact complicates measurement of [FeGEnt]. EcoFepA and KpnIroN bind FeEnt with the same affinity (K_{D}=0.4 nM), but the latter protein also binds FeGEnt (Kd=6.3 nM). As a result, the comparative quenching of EcoFepA-FM and KpnIroN-FM by spent media allowed estimation of its [FeGEnt], as follows. After finding the volume of supernatant required to achieve 50% quenching of 2.5×10⁷ cells of OKN1359/pEcoFepA-FM, we measured the extent of quenching of 2.5×10⁷ cells of OKN1359/pKpnIroN-FM by the same volume of the same supernatant. From the relationship

$$Fr Bd = \frac{1}{\frac{{K}_{{D}_{FeEn t}}}{[ FeEnt ]} + 1} + \frac{1}{\frac{{K}_{{D}_{FeGEnt}}}{[ FeGEnt ]} + 1} = 1 - F / {F}_{0}$$

which expresses the fraction of bound (FrBd) sensor (KpnIroN) to its saturation by both FeEnt and FeGEnt. From experimentally determined F/F₀ and [FeEnt] (=0.4 nM at 50% quenching), we solved for [FeGEnt].

Fluorescence spectroscopic uptake measurements. We employed FD sensors to monitor TonB-dependent uptake of various siderophores by bacterial pathogens. In the fluorescence spectroscopic uptake studies E. coli OKN13 (ΔtonB, ΔfepA) or OKN1359 (ΔtonB, Δfiu, ΔfepA, Δcir) were host strains for pITS23 derivatives carrying single Cys mutants of an LGP of interest. The FLGP were decoy sensors that monitored ferric siderophore uptake by test strains. For each assay we used 10⁷ sensor cells (e.g., OKN13/pFepA-FM (53)) in a 2 mL quartz cuvette containing PBS+0.2% glucose at 37° C. After recording fluorescence intensity for 100 seconds, we added an iron complex (e.g., FeEnt) at a final concentration of ˜10 nM. After incubating the sample for 100 seconds, during which time fluorescence was quenched (e.g., ˜60% quenching for EcoFepA_A698C-FM), we added 0.5-2×10⁷ cells of the test strain (E. coli MG1655, K. pneumoniae Kp52.145, A. baumannii 17978, P. aeruginosa PA01, E. cloacae) and monitored the time-course of fluorescence emissions at 520 nm for 15-40 minutes, with stirring. Transport of the iron complex by the test strain resulted in an increase in fluorescence intensity as the cells depleted it from solution.

Table 1 lists K_{D} values from analysis of fluorescence quenching with ferric or apo (parenthetic values) siderophores or porphyrins. For each determination, we measured the extent of fluorescence quenching of an FLGP, when exposed to sequentially increasing concentrations of the listed metal complexes, as plotted in FIGS. 2-4. We performed each experiment 2 or 3 times. The tabulated K_{D} values are from single, representative experiments, in which we collected each data point in triplicate, plotted F/F₀ vs [ligand] for each quenching titration, and fitted the curves to a single site binding model using Grafit 6.012 (Erithacus Ltd, Middlesex, UK).

In Table 1, the mean standard errors of the K_{D} values from the fitted curves for each FLGP/FBP were: EcoFiu, 6.2%; AbaPiuA, 24%; EcoFepA, 17.3%; KpnFepA1658, 17.3%; KpnFepA4984, 26%; KpnFepA2380, 21%; AbaFepA, 21.1%; PaeFepA, 22.4%; KpnIroN, 26.3%; EcoCir, 13.6%; AbaPirA, 23%; PaeFpvA, 9.7%; EcoFepB, 23%; HsaSCN, 21%; EcoFhuA, 12.1%; EcoIutA, 13%; KpnFyuA, 12%; AbaBauA, 19%; EcoBtuB, 16%; CcrHutA, 18%.

TABLE 1
Affinities (K_{D} values; uM) of LGP sensors for apo and ferric siderophores and porphyrins.

| | | Catecholates | | | | | | | | |
|---|---|---|---|---|---|---|---|---|---|---|
| Sensor¹ | Residue ² | FeEnt | FeEnt* | FeGEnt | FeCrn | FeDHB | FeDHBS | FeFDC | FeMB-1 | FePvd |
| EcoFiu | A694C | 0.55 | 0.46 | 0.4 | NB | NB | 0.10 | 0.54 | NB | NB |
| AbaPiuA | S696C | 4.5 | 2.8 | NB | NB | 12 | NB | 57 | 74 | NB |
| EcoFepA | A698C | 0.0004 | 0.0084 | 0.38 | NB | NB | 1.9 | NB | 37 | NB |
| | | (0.03) | | | | | | | | |
| KpnFepA | A382C | 0.0006 | 0.023 | 0.095 | NB | NB | ND | ND | ND | NB |
| 1658 | | (0.07) | | | | | | | | |
| KpnFepA | A390C | 0.014 | 0.18 | 0.95 | NB | 1.2 | ND | ND | ND | NB |
| 4984 | | (3.3) | | | | | | | | |
| KpnFepA | T255C | 12.8 | NB | NB | 12.8 | 22 | ND | ND | ND | NB |
| 2380 | | | | | | | | | | |
| AbaFepA | S278C | 0.025 | 0.23 | 0.02 | NB | 2.3 | 3.2 | 0.4 | 3.1 | NB |
| | | (1.5) | | | | | | | | |
| PaeFepA | S271C | 0.01 | 0.017 | 0.2 | NB | 2.1 | NB | NB | NB | ND |
| | | (1.7) | | | | | | | | |
| KpnIroN | T210C | 0.0004 | 0.019 | 0.007 | NB | 1.2 | NB | 0.58 | 1.3 | NB |
| | | (0.04) | | | | | | | | |
| EcoCir | G487C | 120 | 7.1 | NB | NB | NB | 8 | 0.06 | 21 | NB |
| AbaPirA | S328C | 9.3 | 1.3 | NB | NB | 188 | NB | 4.8 | 101 | NB |
| | | (7.3) | | | | | | | | |
| PaeFpvA | T660C | 6.9 | 5.6 | NB | NE | 6.8 | NB | NB | NB | 0.004 |
| EcoFepB | T297C | 0.043 | 0.140 | 13 | 9.5 | 5.8 | NB | NB | NB | NB |
| | | (6) | | | | | | | | |
| HsaSCN | Q128C | 0.012 | 0.010 | 0.09 | 0.33 | 0.33 | NB | NB | NB | NB |

| | | Hydroxamates | | | | Mixed Chelation | | | |
|---|---|---|---|---|---|---|---|---|---|
| Sensor¹ | Residue ² | Fc | FcA | FeAbn | FxB | FeYbt | FeAgn | FeFDC | FeMB-1 |
| EcoFhuA | D396C | 0.003 | 0.060 | NB | 100 | NB | NB | NB | NB |
| | | (0.2) | | | | | | | |
| EcoIutA | S549C | NB | NB | 0.006 | NB | NB | NB | NB | NB |
| | | | | (1.4) | | | | | |
| KpnFyuA | S631C | NB | NB | NB | 277 | 0.001 | NB | NB | NB |
| | | | | | | (0.12) | | | |
| AbaBauA | S385C | NB | NB | NB | NB | NB | 1.1 | 181 | 31 |
| HsaSCN | Q128C | 0.11 | NB | NB | 0.175 | 10.5 | 0.16 | NB | NB |

Porphyrins

| Sensor¹ | Residue ² | Hn | B₁₂ |
|---|---|---|---|
| EcoBtuB | S286C | NB | 0.002 |
| CcrHutA | A635C | 0.150 | NB |
| HsaSCN | Q128C | 0.34 | NB |

¹LGP acronyms abbreviate the genus and species of their origin: E. coli FepA, EcoFepA; K. pneumoniae FepA locus 1658, KpnFepA1658; Yersinia pestis FyuA, YpeFyuA; Homo sapien SCN, HsaSCN, etc.
² Location of the Cys substitution in the mature protein sequence.
FeEnt*, partially degraded FeEnt;
NB, no binding;
ND, no data.
K_{D} values of preferred ligands are in bold text.

TABLE 2
Concentrations (μM) of Ent, GEnt, Abn and Ybt in spent bacterial media.

| | | | Strain | | | | |
|---|---|---|---|---|---|---|---|
| FLGP | Ligand | Medium1 | MG1655 | Nissle | CP9 | HvKp1 | HvKp2 |
| EcoFepA | FeEnt | MOPS | 32.5 ± 0.13 | 4.6 ± .01 | 8.4 ± 0.7 | 2.1 ± 0.07 | 1.4 ± 0.07 |
| ″ | ″ | M9 | 3.2 ± 0.1 | 0.04 ± 0.01 | 0.2 ± 0.07 | 0.43 ± 0.03 | 0.267 ± 0.02 |
| KpnIroN | FeGEnt | MOPS | 11.1 ± 1.5 | 71.3 ± 0.2 | 84 ± 0.7 | 6.9 ± 0.07 | 14.9 ± 0.07 |
| ″ | ″ | M9 | 1.6 | 0.3 | 5.1 | 2.8 | 0.9 |
| EcoIutA | FeAbn | MOPS | 0 | 180 ± 14 | 0 | 444 ± 91 | 626 ± 90 |
| ″ | ″ | M9 | 0 | 26.5 ± 0.9 | 0 | 210 ± 11 | 186 ± 17 |
| KpnFyuA | FeYbt | MOPS | 0.01 ± 0.001 | 6.3 ± 0.12 | 19 ± 0.08 | 0.02 ± 0.001 | 1.3 ± 0.23 |
| ″ | ″ | M9 | 0.02 ± 0.001 | 0.8 ± 0.004 | 1.2 ± 0.02 | 0.03 ± 0.001 | 8 ± 0.07 |

In this experiment the cell densities of the six strains (tabulated left-to-right above), after 24 h of growth in iron-restricted M9, were 0.1, 0.18, 0.19, 0.2, 0.44, and 0.43 × 10⁹ cells/mL, respectively.
The densities of the six strains after 24 h of growth in iron-restricted MOPS were 1.25, 1.52, 1.9, 1.56, 1.42 and 1.83 × 10⁹ cells/mL, respectively.
BN1071 was 0 and is omitted from the table.

### Example 2

The sensor technology has been further extended to develop a number of fluorescent binding protein sensors and a general approach for developing new sensor platforms. Membrane transporters and soluble binding proteins recognize particular nutrients, metabolites, vitamins or ligands. By modifying genetically engineered single Cys residues near the active sites of such proteins with extrinsic maleimide fluorophores, one can create sensitive fluorescent sensors that detect, quantify and monitor small molecules that are relevant to the biochemistry, physiology, microbiology and clinical properties of pro- and eukaryotic organisms.

The protocol described focuses on fluorescent modification of engineered Cys residues in the surface loops of Gram-negative bacterial OM proteins (OMPs), to transform them into sensors. Our methodology enables labeling of living cells, which has many advantages. We also describe the labeling of similarly engineered, purified binding proteins (e.g., human siderocalin (HsaSCN). These manipulations transform both types of proteins into sensitive, quantitative biochemical sensors.

### Materials and Reagents

1. Appropriate growth media (e.g., MOPS minimal media)
2. 50 mM NaHPO4, pH 6.75
3. Phosphate-buffered saline (PBS)
4. ˜1 mM fluorescein 5′ maleimide (FM; AnaSpec AS-81405) in anhydrous dimethyl formamide or dimethyl sulfoxide
5. Sensor storage: labeled bacterial cells in media, or purified proteins buffer, respectively, both containing 10% glycerol. Store samples frozen at −70° C.

### Equipment

1. High-speed centrifuge (Beckman-Coulter Avanti J25)
2. Spectrophotometer (Beckman-Coulter DU800)
3. Fluorometer (OLIS Clarity; OLIS-SLM-Aminco 8100,)
4. Typhoon 8600 Biomolecular Imager (GE/Amersham)

### Procedure

### A. Overall Design and Engineering of Site-Directed Fluorescent Sensors.

This methodology is predicated on locating sites in the primary structure of OM or soluble binding proteins that tolerate Cys substitution and chemical modification with extrinsic fluorophores. Using either crystallographic data or structural models (e.g., from the MODELLER algorithm of CHIMERA) we predict accessible sites, engineer single Cys residues at those locations, and subject the resulting Cys mutant proteins to labeling with fluorophores. Regardless of the basis of such predictions, it is important to still experimentally verify the extent of labeling and biochemical functionality of each construct using standard methods. Consequently, for each protein of interest we select 4-6 candidate target residues, mutagenize them to substitute Cysteine, evaluate their expression, their susceptibility to alkylation with different maleimide fluorophores, and their overall sensitivity to interactions with ligands.

### B. Overall Design and Engineering of Site-Directed Fluorescent Sensors.

For a protein whose tertiary or quaternary structure is fully delineated, we pick candidate residues with side chains that project near its ligand binding site, such that adsorption of a ligand may result in the quenching of an attached fluorophore. For example, E. coli FepA (EcoFepA) binds the siderophore ferric enterobactin (FeEnt) in the loops of its surface vestibule. Its crystal structure correctly predicted that Cys mutant proteins EcoFepA_S271C and EcoFepA_A698C are accessible to fluoresceination, and then sensitive to FeEnt binding. For Klebsiella pneumoniae IroN (KpnIroN), on the other hand, that is not yet structurally solved but has 82% identity to EcoFepA, we relied on the guideline that >25% sequence identity predicts an identical overall protein fold. To determine labeling targets in KpnIroN we used CLUSTALW to align it with EcoFepA (PDB sequence 1FEP (Buchanan et al. 1999), and then employed the MODELLER of CHIMERA (UCSF) to predict KpnIroN tertiary structure, including surface loops. This led to the selection and engineering of mutant KpnIroN_T210C, located in L2, that is quantitatively modified by FM.

### C. Cys Mutant Proteins.

As a general approach, we use PCR to clone a binding protein of interest from the chromosomes of a particular bacterial species. When the sensors originate from E. coli OMPs, we precisely clone the nucleotide sequences encoding their mature proteins and insert them downstream from the native promoter of EcofepA in pITS23, which is a derivative of the low-copy pHSG575. One may generate Cys substitution mutants in proteins by a variety of methods; we use QuikChange mutagenesis (Agilent) of the wild type genes on pITS23, with complementary oligonucleotides flanking the mutation, followed by digestion of the wild-type vector by DpnI. After confirming the mutations by sequencing (Genewiz) of purified plasmids, express and fluorescently label the sensor proteins in intact cells, that may be stored frozen at 70° C. If the desired sensor derives from a soluble binding protein, then clone the relevant structural genes in plasmid pET28a, that adds a 6H-tag at either the N- or C-termini of the mature proteins, and purify it by metal ion affinity chromatography.

### D. Cys Mutant Protein Expression and Fluorescent Labeling.

Although other plasmids systems are likely also acceptable, our vector for OMP production, pITS23, carries wild-type EcofepA under control of its native, Fur-regulated promoter. For expression of other E. coli OMP sensors we precisely replace EcofepA in pITS23 with the alternate OMP structural gene (with its own signal sequence), such that the iron-regulated EcofepA promoter controls biosynthesis of the OM protein. For OM proteins of other bacterial species we replace the sequence encoding mature EcoFepA with the sequence encoding the foreign, mature OM protein, downstream from the EcoFepA signal sequence, and regulated by the EcofepA promoter. This approach allows the EcoFepA signal sequence to direct secretion and assembly of the foreign OMP in the E. coli OM. Next, choose appropriate conditions for high-level expression of the OM sensor proteins of interest. We utilize the E. coli host OKN1359, because its inability to make enterobactin (entA) or to transport iron (ΔtonB) causes overexpression of iron-regulated sensor proteins during growth in iron-deficient MOPS media. Additionally, OKN1359 is devoid of several OM proteins (Δfiu, ΔfepA, Δcir). Expose the Cys mutant constructs in situ, in living cells, to FM or other fluorophore maleimides (e.g. coumarin maleimides, Alexa Fluor maleimides, etc.). Evaluate the expression and labeling of each of cloned OMP by growing bacteria harboring the appropriate plasmid construct in MOPS medium to late log phase (A600 nm=2.5-3.5), and analyze SDS-PAGE resolved samples, or perform immunoblots of bacterial OM or soluble purified protein fractions, to visualize the production and purity of the sensor proteins. For soluble binding proteins, purify a 6His-tagged Cys mutant protein from a cell lysate by metal affinity chromatography, modify it with FM or other fluorophore maleimides, and re-purify the fluorescently labeled sensor by acetone precipitation or gel filtration chromatography.

### E. Fluorescence Modifications.

For modifications with fluorophore maleimides, inoculate bacteria harboring plasmids that encode a Cys mutant OMP from frozen stocks into LB, grow the strain overnight and sub-culture at 1% into MOPS minimal media, with shaking (200 rpm) at 37° C. for 10-12 h, until late exponential phase. After collecting the cells by centrifugation at 7500×g for 15 min, and washing with 50 mM NaHPO4, pH 6.7, label the Cys-mutant OMP with 5 μM FM in 50 mM NaHPO4, pH 6.7 for 15 min at 37° C.; terminate the labeling reactions by adding β-mercaptoethanol to 140 μM. After collecting the fluoresceinated cells by centrifugation at 7500×g for 15 min, and washing with 50 mM NaHPO4, pH 6.7, re-suspended the labeled bacteria in phosphate-buffered saline (PBS). Use the labeled cells immediately in spectroscopic experiments, or rest them on ice (up to 24 h), or add glycerol to 15% and store them (indefinitely) as 1 mL aliquots at −70° C. In the latter case, after thawing the labeled cells pellet them by centrifugation in a microfuge, wash them once with and re-suspended them in PBS. For evaluation of protein expression or the extent of FM-labeling, solubilize aliquots of the cell suspensions with sample buffer, and subject them to SDS-PAGE. After electrophoresis, first visualize the extent of fluorescence labeling on a Typhoon 8600 Biomolecular Imager (GE/Amersham), and then stain the gels with Coomassie blue R (Ames, 1974).

### F. Fluorescence Spectroscopic Binding Determinations.

We observe fluorophore-labeled cells in an OLIS-SLM AMINCO 8100 fluorescence spectrometer, upgraded with an OLIS operating system and software (OLIS SpectralWorks, OLIS Inc., Bogart, GA), to control its shutters, polarizers and data collection. We also utilize an OLIS Clarity fluorescence spectrometer, with the same operating software, for fluorescence assays. For binding determinations deposit 2.5×10⁷ labeled cells in a quartz cuvette (final volume, 2 mL) with stirring at 37° C., measure the initial fluorescence (F₀), and then add increasing concentrations of a ligand while monitoring the quenching of fluorescence emissions (F) at 520 nm.

### Results

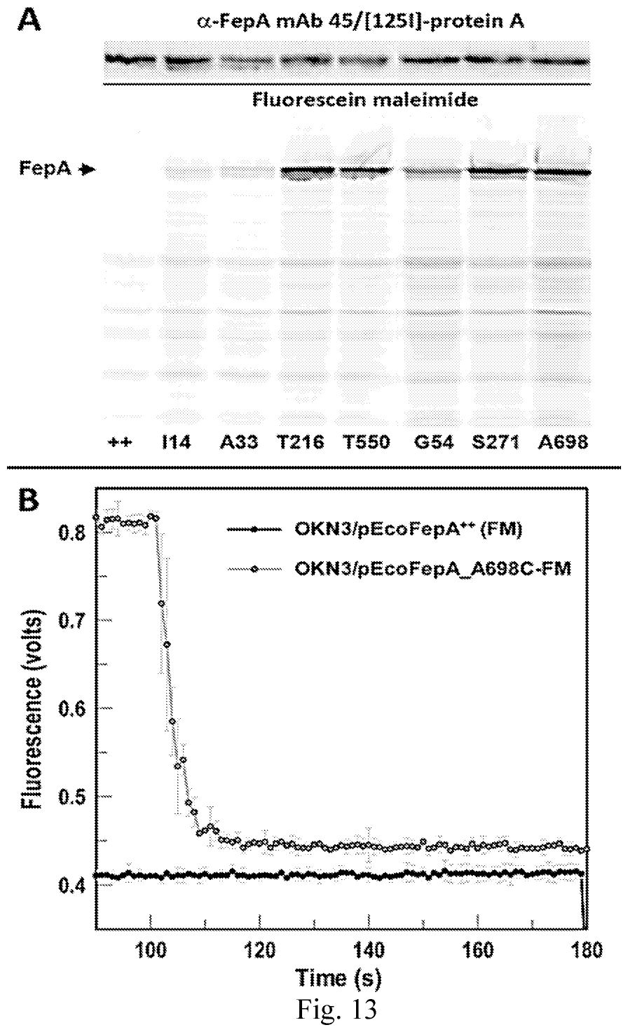

Representative data is shown in FIG. 13. For panel A, Wild-type (++) EcoFepA and its 7 Cys mutants were grown in MOPS media, labeled with 5 μM FM in 50 mM NaHPO4, pH 6.7, for 15 min at 37° C. Cell lysates were resolved by SDS-PAGE (Ames, 1974) and the gel was scanned for fluorescence at 520 nm on a Typhoon imager. FM did not react with wild-type FepA, but it labeled the Cys mutants to different extents. The gel proteins were transferred to nitrocellulose and probed with α-FepA mAb 45 (Murphy et al, 1990)/[¹²⁵I]-protein A to monitor expression. Certain mutants (T216C, S271C, A698C) were stoichiometrically labeled by FM. Panel B shows analysis of fluorescence quenching during FeEnt binding to EcoFepA-A698C-FM. Cells (OKN3 (ΔfepA (Ma et al., 2007)) producing EcoFepA or its mutant A698C were exposed to FM, washed and assayed in an OLIS/SLM-Aminco 8100 fluorometer, with excitation at 488 nm and emission at 520 nm. EcoFepA_A698C-FM showed intense fluorescence that was quenched when 10 nM FeEnt was added at 100 s. Data points are the means of triplicate measurements; grey error bars represent the associated standard deviations of means.

### Protocol

1. Grow sensor strains to maximize Cys-mutant OMP expression, or purify soluble Cys-mutant binding proteins.
2. For bacteria, determine cell density from O.D. 600 nm; determine the concentration of purified proteins from absorbance at 280 nm.
3. Pellet bacterial cells by centrifugation at 7500×g for 15 min and resuspend in the same volume of 50 mM NaHPO4, pH 6.7. Repeat. For soluble binding proteins, dialyze the solution of purified protein (1-5 mg/mL) overnight against 50 mM NaHPO₄, pH 6.7.
4. Dissolve ˜1 mg of FM in 0.5 mL of anhydrous dimethyl formamide or dimethyl sulfoxide; determine [FM] by measuring the absorbance of a 1/100 dilution in 10 mM Tris-Cl, pH 8 (ε^{mM}_{488 nm}=8.1).
5. Covalently modify bacterial cells or purified proteins with 5 μM FM (or other maleimide fluorophore) in 50 mM NaHPO₄, pH 6.7, for 15 min, and quench the reaction with 140 μM β-mercaptoethanol
6. Pellet bacterial cells by centrifugation at 7500×g for 15 min and re-suspend in the same volume of PBS. Repeat the centrifugation and resuspend the cells in PBS at ˜10⁹/mL; dialyze fluoresceinated soluble binding proteins against PBS overnight to remove excess fluorophore.
7. Assess the efficacy of covalent modification by diluting aliquots of fluoresceinated cells or proteins in PBS in a fluorometer; analyze with excitation at 488 nm and emission at 520 nm. Calculate the specific fluorescence of the samples (fluorescence intensity/10⁹ cells or/mg protein).
8. Measure the extent of fluorescence quenching by natural ligands.
9. Use, store on ice (up to 24 h) or preserve at −70° C. in 10% glycerol.

### Data Analysis

For fluorescence spectroscopic measurements of sensor-ligand quenching, perform each measurement in triplicate and calculate the mean value of F/F₀ at each ligand concentration. For analysis and curve-fitting of fluorescence quenching data, plot 1-F/F₀ vs [ligand] and analyze the data by a 1-site binding model (using Grafit 6.0.12 (Erithacus Ltd. West Sussex, UK) or Enzfitter (Biosoft, Cambridge, UK), that fits data to a single site saturation curve, where the amount of ligand bound is plotted as a function of the amount free:

$$[ Boun d ] = \frac{C apacity [ Free ]}{{K}_{D} + [ F r e e ]}$$

These plots yield K_{D} values for each of the receptor-ligand interactions, with associated standard errors.

### Notes

### 1. Host Bacteria.

OM proteins from many Gram-negative bacteria, including E. coli, K. pneumoniae, Acinetobacter baumannii, Caulobacteri crescentus and more, are susceptible to FM labeling in situ, in the cell envelopes of those living cells. But the cell surface of laboratory E. coli is a preferred environment for expression and labeling of both native and foreign OM proteins, because it is not obscured by an LPS O-antigen nor capsular polysaccharide. Hence, we perform most labeling reactions in laboratory E. coli host strains.

### 2. Protein Expression.

It's advisable to adopt a cloning/expression strategy that maximizes production of the target OM protein. We use iron-regulated Fur promoters in conjunction with iron-deficient MOPS media to maximize the expression of foreign proteins in E. coli. However, other promoters (e.g., lac or tac) are equally effective.

### 3. Media for Bacterial Growth and Target Protein Expression.

As a result of the iron-regulated promoters in our experiments, we utilize MOPS minimal media, that is a complete minimal media for Enterobacteriaceae, and readily rendered iron-deficient by excluding FeSO₄ and tricine from its formulation. We have no experience with the chemical modification of cells grown in other defined medias or broths, but we anticipate that regardless of growth media, preliminary washing with 50 mM NaHPO₄, pH 6.7 will lead to efficacious maleimide labeling reactions.

### Recipes

### 1. Iron-Deficient MOPS Minimal Medium

### a. 10×MOPS Concentrate.

The following recipe is for 1 L OF 10× concentrate.

| | |
|---|---|
| MOPS | 83.7 g |
| NH₄Cl | 5.1 g |
| K₂SO₄ | 0.48 g |
| CaCl₂ | 0.56 mg |
| MgCl_{2.}6H₂O | 1.07 g |
| NaCl | 29.25 g |

Add MOPS to about 800 mL of double distilled H₂O. Adjust pH to 7.4 with KOH pellets (˜14 g), add the other components, bring the volume to 1 L and pass through a 0.2-micron filter. DO NOT AUTOCLAVE.

### b. 1000× Micronutrient Solution:

The amounts are for 100 ml of 1000× micronutrient solution.

| | |
|---|---|
| (NH₄)₆Mo₇O₂₄-4H₂O | 37 mg |
| CoCl₂-6H₂O | 71 mg |
| HBO₃ | 250 mg |
| CuSO₄-5H2O | 25 mg |
| MnCl₂ | 160 mg |
| ZnCl₂ | 13.6 mg |

Pass through a 0.2-micron filter. DO NOT AUTOCLAVE. Dilute 1:1000 into final media

c. 1000× KH₂PO₄ Solution:

30 g in 100 ml. Dilute 1:1000 into the final media

d. To prepare the final media.

Add 1 ml of KH₂PO₄ solution to 900 ml of d²H₂O in an iron-free flask and autoclave. Add 100 ml of filter-sterilized 10 MOPS concentrate and 1 ml of filter-sterilized 1000× micronutrient solution. Before inoculation, add a carbon source (e.g., 0.4% glucose), amino acids and vitamins for auxotrophic markers, and appropriate antibiotics.

### 2. Iron-Free Glassware.

To remove adventitious iron from glassware, fill the flasks or bottles with 0.1N HCl, soak overnight at room temperature, and thoroughly rinse with distilled water.

### 3. 50 mM Sodium Phosphate, pH 6.7.

a. 6.9 g Sodium dihydrogen phosphate monohydrate (NaH₂PO₄*H₂O) in 1 L distilled water
b. adjust pH to 6.7 with 1 M NaOH.
   4. Phosphate-Buffered Saline. Dissolve the Following in 1 L Distilled Water:

| | |
|---|---|
| NaCl | 8 g |
| KCl | 0.2 g |
| Na₂HPO₄ | 1.44 g |
| KH₂PO₄ | 0.24 g |

### Example 3

### Enzyme Sensor

Penicillins, ampicillin, and carbapenems are members of the general class of antibiotics called β-lactams, which inhibit a specific cell envelope biosynthetic reaction, and thereby kill the target bacterium. Penicillinases/β-lactamases/carbapenemases are a bacterial defense mechanism against β-lactam antibiotics, that make the target bacterium antibiotic-resistant. Carbapenemases, in particular, are β-lactamases with versatile hydrolytic capacities. They are produced mainly in Gram-negative bacteria, and confer the largest antibiotic-resistance spectrum of concern among bacteria, because they can hydrolyze not only carbapenems but all of the β-lactam antibiotics, including penicillins, cephalosporins, monobactams, and cephamycins. Carbapenemase production can be intrinsic to the bacterial species, but more concerning, can be acquired by bacteria via acquisition of a plasmid containing the genes encoding the carbapenemase. Bacteria producing these β-lactamases may cause serious infections in which the carbapenemase activity renders many β-lactam treatments ineffective. The five most clinically relevant carbapenemases, also known as the “Big Five,” are: Klebsiella pneumoniae carbapenemase (KPC); the metallo-beta-lactamases of Imipenemase (IMP), New Delhi (NDM), and Verona integron-encoded (VIM) groups; and Oxacillin carbapenemases (OXA). Penicillinase/β-lactamase/carbapenemase sensor detects the presence of β-lactam antibiotics, that bind to the sensor protein (fluorescently labeled E. coli transpeptidase Pbp2), and quench its fluorescence emissions.

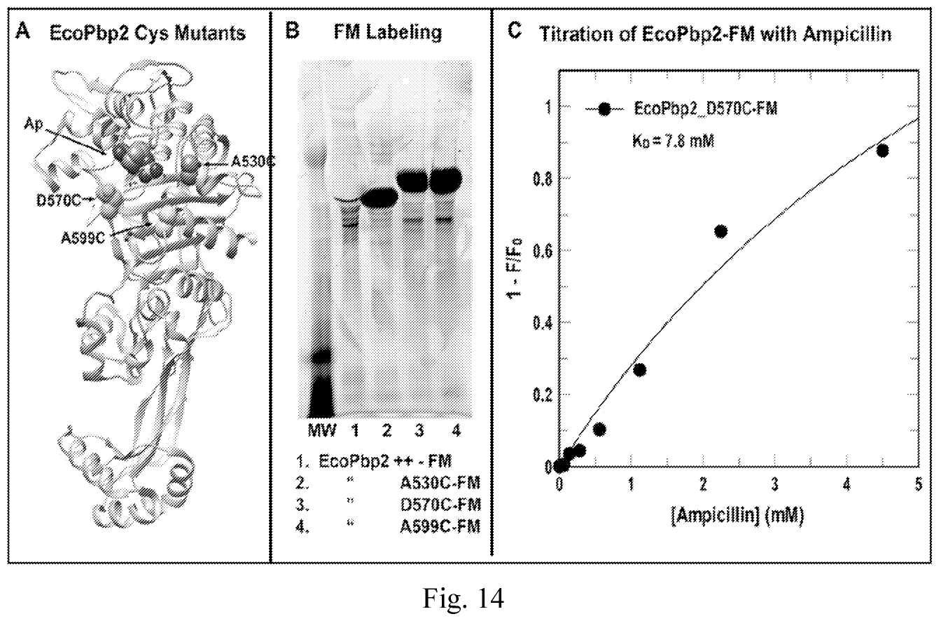

Thus, it is relevant to develop sensors to detect such organisms in samples. Initial studies have been conducted and the results are shown in FIG. 14. As shown in FIG. 14 panel A, from the crystal structure of EcoPbp2 (PDB 6G9F) we selected residues A530, S570 and A599, that surround the active site of the enzyme, for substitution to Cysteine. Panel B shows that after mutagenesis and verification of the resulting mutants by DNA sequencing, we purified the proteins and labeled them with fluorescein maleimide (FM). All three mutants were strongly fluorescently labeled, whereas the wild-type protein was not labeled. As shown in panel C, the mutant D570C-FM was strongly quenched by binding of ampicillin (Ap), and we titrated the protein with increasing concentrations of Ap to determine its affinity for the β-lactam antibiotic: K_{D}=7.8 mM. These data demonstrate the ability of the engineered sensor, EcoPbp2_D570C-FM, to detect and quantify β-lactam antibiotics in solution.

Thus, a penicillinase/β-lactamase/carbapenemase sensor developed using these same approaches can detect the presence of β-lactam antibiotics, that bind to the sensor protein (fluorescently labeled E. coli transpeptidase Pbp2), and quench its fluorescence emissions, as demonstrated in FIG. 14, where ampicillin is used to quench EcoPbp2_D570C-FM. From the extent of the quenching phenomenon, we can detect the presence of the target molecule, and determine its concentration. Interpretation the of proposed penicillinase/β-lactamase/carbapenemase sensor is slightly different because the binding protein is an enzyme that normally participates in cell envelope biosynthesis. However, using the same mechanics as exemplified above, we can utilize EcoPbp2-FM as sensor for such enzymes (i.e., penicillinases, carbapenemases, or other β-lactamases) by adding the putative enzyme-containing sample to the EcoPbp2-FM-ampicillin binding reaction. If a sample contains the target enzyme (e.g., because the sample likewise contains a bacterium secreting this protein), then the ampicillin added to the assay solution will be degraded, and the extent of quenching of EcoPbp2-FM will be proportionally reduced according to the amount of enzyme that is present (and indirectly, the amount of ampicillin that it degrades). So, in this sensor platform, the less quenching in the EcoPbp2-FM-ampicillin binding reaction is diagnostic for the presence of the target enzymes (e.g., a β-lactamase) in the sample, and correspondingly, the presence of a microbe secreting this antibiotic-resistance enzyme. It will be appreciated that the faster a clinician can identify the presence of particular antibiotic resistance infectious agents, the faster the clinician can start the patient on an alternative treatment plan and avoid poor outcomes.

1. An in vitro method for detecting a microbe in a biological sample, said method comprising:
providing an assay solution comprising a high affinity protein-based sensor, wherein the high affinity protein-based sensor comprises a high affinity binding protein engineered with a detectable label that generates a detectable signal;
exposing said assay solution to an energy source to generate said detectable signal;
adding a biological sample containing or suspected of containing said microbe to said assay solution, wherein said high affinity binding protein is specific for a microbe-associated compound secreted or produced by said microbe;
exposing said assay solution to an energy source and detecting a change in the detectable signal in the assay solution over time, wherein said changes correspond to interaction of said microbe-associated compound with said high affinity binding protein.

2. The method of claim 1, wherein said microbe-associated compound binds with said high affinity binding protein, wherein said change in the detectable signal is quenching of said detectable signal.

3. The method of claim 2, wherein speed, duration, or intensity of the quenching indicates activity or concentration of said microbe in said sample.

4. The method of claim 1, wherein said detectable label is a fluorophore.

5. The method of claim 4, wherein said fluorophore is a maleimide fluorophore.

6. The method of claim 1, wherein said microbe-associated compound is a metal or metalated complex.

7. The method of claim 1, wherein said microbe-associated compound is selected from the group consisting of ferric catecholates, ferric hydroxamates, mixed iron complexes, and porphyrins.

8. The method of claim 1, wherein said microbe-associated compound is selected from the group consisting of enterobactin, degraded enterobactin, glucosylated enterobactin, dihydroxybenzoate, dihydroxybenzoyl serine, cefidericol, MB-1, ferrichromes, aerobactin, yersiniabactin, acinetobactin, pyoverdine, hemin, vitamin B12, enzymes, and toxins.

9. The method of claim 1, wherein said biological sample is selected from the group consisting of bodily fluids, food and drink samples, environmental samples, and research materials.

10. The method of claim 1, wherein said biological sample is selected from the group consisting of blood, plasma, serum, urine, exhaled breath condensate, sputum, bronchoalveolar lavage fluid, sweat, saliva, cervicovaginal fluid, rectal secretion, tears, dairy milk, meat, food or drink ingredients, food or drink pre-mixes, food processing equipment swab sample, food handling equipment swab sample, soil samples, water samples, plant tissue, water, cell culture media.

11. The method of claim 1, wherein said high affinity protein-based sensor is a transport-deficient bacterial cell expressing said engineered high affinity binding protein.

12. The method of claim 11, wherein said transport-deficient bacterial cell is a Gram-negative bacteria selected from the group consisting of Acinetobacter baumannii, Klebsiella pneumoniae, Pseudomonas aeruginosa, Enterobacter aerogenes, Caulobacter crescentus, and Escherichia coli.

13. The method of claim 1, wherein said microbe-associated compound is an enzyme, said method further comprising adding a substrate of said enzyme to said assay solution along with said biological sample, wherein quenching of said detectable signal is reduced if said microbe is present in said biological sample.

14. The method of claim 1, wherein said high affinity binding protein comprises an amino acid residue that has been engineered with a detectable label.

15. The method of claim 14, wherein said engineered amino acid residue consists of a cysteine substitution, wherein said detectable label is attached to said cysteine residue.

16. The method of claim 1, wherein said high affinity binding protein is selected from the group consisting of EcoFiu, EcoFepA, EcoFepB, EcoCir, EcoFhuA, EcoIutA, EcoBtuB, KpnIroN, KpnFepA, KpnFyuA, AbaPiuA, AbaFepA, AbaPirA, AbaBauA, PaeFepA, PaeFpvA, CcrHutA, and HsaSCN, engineered with a cysteine substitution and detectable label attached to said cysteine residue.

17. The method of claim 1, wherein said assay solution has a volume of less than about 300 μL total solution.

18. The method of claim 1, further comprising distributing said assay solution into a plurality of individual reaction vessels before adding said biological sample, each vessel comprising respective reaction volumes.

19. The method of claim 18, wherein each reaction vessel is a microwell in a multi-compartment microplate.

20. The method of claim 19, wherein a plurality of different assay solutions comprising different high affinity protein-based sensors, are added to respective microwells on said microplate, each microwell containing a different assay solution.

21. The method of claim 20, where each of said different high affinity protein-based sensors has a binding affinity for a different a microbe-associated compound, said method further comprising adding aliquots of said biological sample to each of said microwells, and detecting changes in the detectable label in each microwell, wherein said changes indicate activity of a respective microbe in said microwell.

22. A kit for detecting a microbe in a biological sample, said kit comprising:
a vessel containing a high affinity protein-based sensor, wherein the high affinity protein-based sensor comprises a high affinity binding protein engineered with a detectable label that generates a detectable signal, wherein said high affinity binding protein is specific for a microbe-associated compound secreted or produced by said microbe;
instructions for creating an assay solution with said high affinity protein-based sensor;
instructions for exposing said assay solution to an energy source to generate said detectable signal;
instructions for adding a biological sample containing or suspected of containing said microbe to said assay solution and detecting changes in the detectable signal in the assay solution over time to detect the interaction of said microbe-associated compound with said high affinity binding protein.

23. The kit of claim 22, wherein high affinity protein-based sensor is cryopreserved, said kit further comprising instructions for thawing and reconstituting said sensor to creating said assay solution.

24. The kit of claim 22, wherein said high affinity protein-based sensor is a transport-deficient bacterial cell expressing said engineered high affinity binding protein.

25. The kit of claim 22, wherein said vessel is a microwell in a multi-compartment microplate.

26. A device for detecting a microbe in a biological sample, said device comprising a multi-compartment microplate comprising a plurality of microwells, further comprising a high affinity protein-based sensor deposited in one or more of said microwells.

27. The device of claim 26, said device comprising a high affinity protein-based sensor deposited in two or more of said microwells.

28. The device of claim 26, said device comprising a high affinity protein-based sensor deposited in a plurality of said microwells.

29. The device of claim 26, each of said microwells comprising a respective high affinity protein-based sensor deposited therein.

30. The device of claim 27, said device comprising two or more different high affinity protein-based sensor deposited in respective microwells, wherein each high affinity protein-based sensor has a binding affinity for a different microbe-associated compound.

31. The device of claim 27, said device comprising a plurality of different high affinity protein-based sensor deposited in respective microwells, wherein each high affinity protein-based sensor has a binding affinity for a different microbe-associated compound.
